(19) United States
(12) Patent Application Publication
(10) Pub. No.: US 2026/0256944 A1
(43) Pub. Date: Sep. 3, 2026

(54) AAV-SGSH Vectors for Treatment of Mucopolysaccharidosis IIIA

(71) Applicant: The University of North Carolina at Chapel Hill, Chapel Hill, NC (US)

(72) Inventors: Haiyan Fu, Durham, NC (US); Tierra Bobo, Mebane, NC (US)

(21) Appl. No.: 18/871,527

(22) PCT Filed: Jun. 9, 2023
(86) PCT No.: PCT/US2023/068176

§ 371 Date: Dec. 4, 2024

Related U.S. Application Data
(60) Provisional application No. 63/350,541, filed on Jun. 9, 2022.

(51) Int. Cl. A61K 48/00; A61P 25/28; C12N 9/14; C12N 15/86

(52) U.S. Cl. CPC A61K 48/005; A61P 25/28; C12N 9/14; C12N 15/86; C12N 2750/14143; C12N 2750/14151; C12N 2800/22; C12N 2830/50; C12Y 310/01001

(57) ABSTRACT

This invention relates to viral vectors for delivery of N-sulfoglucosamine sulfohydrolase (SGSH) to a subject. In some aspects, the SGSH sequence is optimized for expression in human cells. The invention further relates to methods of using the vector to increase secretion of SGSH from a cell and for treatment and prevention of mucopolysaccharidosis IIIA.

### STATEMENT OF PRIORITY

This application claims the benefit, under 35 U.S.C. § 119 (e), of U.S. Provisional Application No. 63/350,541, filed Jun. 9, 2022, the entire contents of which are incorporated by reference herein in their entirety.

### STATEMENT REGARDING ELECTRONIC FILING OF A SEQUENCE LISTING

A Sequence Listing in XML format, entitled 5470-931WO_ST26.xml, 40,560 bytes in size, generated on Jun. 1, 2023 and filed herewith, is hereby incorporated by reference in its entirety for its disclosures.

### FIELD OF THE INVENTION

This invention relates to viral vectors for delivery of N-sulfoglucosamine sulfohydrolase (SGSH) to a subject. In some aspects, the SGSH sequence is optimized for expression in human cells. The invention further relates to methods of using the vector to increase secretion of SGSH from a cell and for treatment and prevention of mucopolysaccharidosis IIIA.

### BACKGROUND OF THE INVENTION

Mucopolysaccharidosis (MPS) IIIA is a devastating lysosomal storage disease (LSD) with severe neuropathy. The disease is caused by autosomal recessive mutations in N-sulfoglucosamine sulfohydrolase (SGSH), a lysosomal enzyme that is essential for the degradation of a class of biologically important glycosaminoglycans (GAGs), heparan sulfate (Neufeld & Muenzer (2001) The Metabolic & Molecular Basis of Inherited Disease (eds. Scriver, et al.) 3421-3452, McGraw-Hill, New York; St Louis; San Francisco; Freeman & Hopwood (1986) Biochem. J. 234:83-92). While the mutations are highly heterogeneous, the lack of functional SGSH results in lysosomal GAG storage in cells in virtually all organs, leading to multisystem manifestations with profound neuropathologies throughout the nervous system (Neufeld & Muenzer (2001) The Metabolic & Molecular Basis of Inherited Disease (eds. Scriver, et al.) 3421-3452, McGraw-Hill, New York; St Louis; San Francisco; Yogalingam & Hopwood (2001) Hum. Mutat. 18:264-81). Infants with MPS IIIA appear normal at birth. The majority of MPS IIIA patients are diagnosed before age 6 years, when severe neurological disorders have developed. Premature death typically occurs in the second decade (Neufeld & Muenzer (2001) The Metabolic & Molecular Basis of Inherited Disease (eds. Scriver, et al.) 3421-3452, McGraw-Hill, New York; St Louis; San Francisco; Yogalingam & Hopwood (2001) Hum. Mutat. 18:264-81; Valstar, et al. (2008) J. Inherit. Metab. Dis. 31 (2): 240-52). No treatment is currently available; palliative care has been the only option for MPS IIIA. The blood-brain-barrier (BBB) has been the major challenge to therapeutic development for neuropathic LSDs.

Gene therapy offers an ideal strategy for treating the majority of LSDs by targeting the root-cause, with the potential for long-term endogenous expression of functional recombinant enzymes by replacing the defective gene. Given the bystander effects of lysosomal enzymes, there is no need to transduce every cell to achieve the optimal therapeutic benefits. While numerous viral vectors have been studied targeting different LSDs, recombinant adeno-associated virus (rAAV) vectors have been the favored tools for gene delivery because of its safe profiles, long-term transgene expression, and diverse cell and tissue tropisms of different AAV serotypes (Daya & Berns (2008) Clin. Microbiol. Rev. 21:583-93; Zincarelli, et al. (2008) Mol. Ther. 16:1073-80; Samulski, et al. (1987) J. Virol. 61:3096-3101; Berns & Linden (1995) Bioessays 17:237-245). The demonstrated trans-BBB-neurotropic AAV9 (Zincarelli, et al. (2008) Mol. Ther. 16:1073-80; Foust, et al. (2009) Nat. Biotechnol. 27:59-65; Duque, et al. (2009) Mol. Ther. 17:1187-96) has offered a great gene delivery tool for the treatment of monogenic diseases with neurological manifestations.

The present invention addresses unmet needs by providing improved therapeutic efficacy. The invention provides improved viral vectors for expression of SGSH in the CNS and methods for treating or preventing MPS IIIA.

### SUMMARY OF THE INVENTION

Previously, the inventors developed a first-generation gene therapy product using rAAV9 vector to deliver the human SGSH gene (hSGSH) cDNA driven by a murine small nuclear RNA ula promoter via systemic delivery, leading to IND approval for a Phase I/II gene therapy clinical trial in patients with MPS IIIA (Fu, et al. (2016) Mol. Ther. Methods Clin. Dev. 3:16036). In addition, a self-complementary adeno-associated virus 9 (scAAV9) vector was generated to deliver the wild-type hSGSH gene by a single intravenous (i.v.) infusion (Bobo, et al. (2020) Mol. Ther. Methods Clin. Dev. 19:474-485). The present invention is based on the finding that the use of AAV vectors comprising a nucleic acid encoding SGSH that is codon-optimized for expression in human cells provides an unexpected increase in both expression and secretion of SGSH. These vectors can be used advantageously for treatment of MPS IIIA as the treatment may be more effective than previous vectors for the dual reasons of enhanced expression levels in infected cells and increased bystander effect in non-infected cells due to enhanced secretion.

Thus, one aspect of the invention relates to a recombinant nucleic acid comprising a sequence encoding human N-sulfoglucosamine sulfohydrolase (SGSH) that is codon-optimized for expression in human cells, wherein the recombinant nucleic acid comprises a nucleotide sequence at least 90% identical to SEQ ID NO:1.

Another aspect of the invention relates to an AAV vector genome comprising the nucleic acid of the invention, an AAV particle comprising the AAV vector genome, and a pharmaceutical composition comprising the AAV particle.

A further aspect of the invention relates to a method of producing a recombinant AAV particle comprising an AAV capsid, the method comprising: providing a cell in vitro with an AAV Cap and AAV Rep coding sequences, the AAV vector genome of the invention, and helper functions for generating a productive AAV infection; and allowing assembly of the recombinant AAV particle comprising the AAV capsid and encapsidating the AAV vector genome.

An additional aspect of the invention relates to a method of expressing SGSH in a cell, comprising contacting the cell with an effective amount of an AAV particle of the invention, thereby expressing SGSH in the cell.

Another aspect of the invention relates to a method of increasing secretion of SGSH from a cell, comprising contacting the cell with an effective amount of the AAV particle of the invention, thereby increasing secretion of SGSH from the cell relative to the secretion of SGSH after contacting the cell with an AAV particle comprising a nucleic acid comprising the wild-type sequence for SGSH.

A further aspect of the invention relates to a method of delivering SGSH to a subject, comprising administering to the subject an effective amount of the AAV particle or the pharmaceutical formulation of the invention, thereby delivering SGSH to the subject.

An additional aspect of the invention relates to a method of treating or delaying the onset of mucopolysaccharidosis IIIA (MPS IIIA) in a subject in need thereof, comprising administering to the subject a therapeutically effective amount of the AAV particle or the pharmaceutical formulation of the invention, thereby treating or delaying the onset of MPS IIIA in the subject.

Another aspect of the invention relates to use of the AAV particle or the pharmaceutical formulation of the invention to treat or delay the onset of MPS IIIA.

A further aspect of the invention relates to use of the AAV particle or the pharmaceutical formulation of the invention in the preparation of a medicament to treat or delay the onset of MPS IIIA.

These and other aspects of the invention are set forth in more detail in the description of the invention below.

### BRIEF DESCRIPTION OF THE DRAWINGS

FIG. 1 shows the structures of scAAV-mCMV-hSGSH and scAAV-mCMV-hSGSH^{op} viral vector genomes. ITR: wild-type AAV2 terminal repeat; dTR: AAV2 terminal repeat with deletion of terminal resolution site to force generation of self-complementary dimeric genomes; mCMV: a truncated 228 bp mini CMV promoter; hSGSH: human N-sulfoglucosamine sulfohydrolase coding region; hSGSH^{op}: codon-optimized hSGSH coding region; Poly A: SV40 polyadenylation signal.

FIGS. 2A-2C show AAV-hSGSH^{op}-mediated effective expression and enhanced secretion of rSGSH in vitro. HEK 293 cells were transfected in duplicate with 1 μg plasmid of ptrs-mCMV-hSGSH (SGSH) or ptrs-mCMV-hSGSH^{op} (SGSG^{op}). Controls were non-transfected cells (NT). Media (FIG. 2A) and cell lysates (FIG. 2B) were assayed in duplicate for SGSH activity at 48 h post-transfection. FIG. 2C. Total SGSH activity (cell +media).

FIGS. 3A-3D show correction of behavior deficits and extension in survival. MPS IIIA mice were treated with an IV injection of scAAV9-mCMV-hSGSH vector at 2.5×10¹² vg/kg or 5×10¹² vg/kg at age 1 m (FIG. 3A), or 1×10¹³ vg/kg at age 3 m (FIG. 3B) or 6 m (FIG. 3C). FIGS. 3A-3D: Behavior performance was tested in a hidden task in Morris water maze at age 8 m and/or 12 m. RT: mice treated at age 3 m and 6 m were re-tested at age 12 m. Wild-type (WT) and non-treated MPS IIIA (IIIA) mice were used as controls. m/m: injection age/testing age (time); *: P<0.05 vs. IIIA; \hat{ }: P>0.05 vs. IIIA; ^{#}: P>0.05 vs. WT. FIG. 3D. Survival.

FIGS. 4A-4D show persistent restoration of SGSH activity in CNS and somatic tissues. MPS IIIA mice were treated at age 1 m with an IV injection of 2.5×10¹² vg/kg (FIG. 4A) or 5×10¹² vg/kg (FIG. 4B), or at 3 m or 6 m with 1×10¹³ vg/kg (FIG. 4C) scAAV9-mCMV-hSGSH vector. FIGS. 4A-4C. Tissues were assayed for SGSH activity at 1 m post-injection (pi) and age 8 m (n=4-5/group) or humane endpoint (n=5-9). SGSH activity is expressed as units/mg protein, 1 unit=nmol of 4MU released/17 hr. *: P≤0.05 vs. WT; #: P>0.05 vs. WT. Note: There is <3% of normal SGSH activity in tissues in MPS IIIA mice. FIG. 4D. Tissues were assayed by immunofluorescence for hSGSH. rSGSH-positive signals and autofluorescence signals were detected. NT: nontreated MPS IIIA mice; AAV9: AAV9-treated MPS IIIA mice (5×10¹² vg/kg, injected at age Im, assayed at 7 m pi); CTX: cerebral cortex; BS: brain stem; HP: hippocampus; Liv: liver; Hrt: heart; Kid: kidney; Spl: spleen; Int: Intestine; Open arrows: autofluorescent red blood cells; Closed arrows: hSGSH-positive brain cells; Asterisk: hSGSH-positive blood vessels; Arrow heads: glomerulus.

FIGS. 5A-5D show diminishment of lysosomal storage pathology and astrocytosis. MPS IIIA mice were treated at age 1 m with an IV injection of scAAV9-mCMV-hSGSH vector at doses of 2.5×10¹² vg/kg (FIG. 5A), 5×10¹² vg/kg (FIG. 5B), and 1×10¹³ vg/kg (FIG. 5C). FIGS. 5A-5C: Tissues were assayed for GAG contents at 1 m pi, age 8 m, or humane endpoint. GAG content is expressed as ug/mg wet tissue. m/m: injection age/testing time. *: p<0.05 vs. WT; #: p>0.05 vs. WT; \hat{ }: p≤0.05 vs. IIIA. FIG. 5D. Immunofluorescence for LAMP1 or GFAP (5×10¹² vg/kg, injected at age 1 m, assayed at 7m pi). CTX: cerebral cortex; BS: brain stem; Liv: liver; Hrt: heart; Ret: retina; Msc: muscle; Kid: kidney; Spl: spleen; Int: Intestine; Open arrows: inner surface; Closed arrows: choroid; NT: non-treated MPS IIIA; AAV9: vector treated MPS IIIA.

FIGS. 6A-6C show biodistribution of systemically delivered scAAV9-mCMV-hSGSH in MPS IIIA mice. MPS IIIA mice were treated by an IV injection of scAAV9-mCMV-hSGSH at age 1 m at 2.5×10¹² vg/kg (FIG. 6A) or 5×10¹² vg/kg (FIG. 6B), or 1×10¹³ vg/kg at age 3 m or 6 m (FIG. 6C). Tissues were assayed in duplicate by qPCR at 1 m pi, age 8 m or humane endpoint (n=4-9/group). Data expressed as 105 vector genome (vg) per μg genomic DNA (gDNA). *Vector genome was detected at <0.005×10⁵ vg/ug gDNA in non-treated WT and MPS IIIA mice. m/m: injection age/testing time.

FIG. 7 shows restoration of SGSH activity in CNS and somatic tissues following scAAV9-hSGSH^{op} gene delivery. MPS IIIA mice were treated at age 1-2 m with an IV injection of 5×10¹² vg/kg, 1×10¹³ vg/kg or 2×10¹³ vg/kg, or with combined IV (2×10¹² vg/kg) and IT injection (1×10¹² vg/kg). Tissues were assayed for SGSH activity at 1 m pi (n=4/group). SGSH activity is expressed as units/mg protein, 1 unit=nmol of 4MU released/17 hr. WT: wild-type mice. Liv: liver; Spl: spleen; Int: intestine; Hrt: heart; Kid: kidney; Mus: skeletal muscle.

FIG. 8 shows clearance of GAG storage in the CNS and peripheral tissues following scAAV9-hSGSH^{op} gene delivery. MPS IIIA mice were treated at age 1-2 m with an IV injection of 5×10¹² vg/kg, 1×10¹³ vg/kg or 2×10¹³ vg/kg, or with combined IV (2×10¹² vg/kg) and IT injection (1×10¹² vg/kg). Tissues were assayed for GAG contents at 1 m pi (n=4/group). GAG content is expressed as ug/mg wet tissue. WT: wild-type mice; IIIA: non-treated MPS IIIA mice. *: p≤0.05 vs. IIIA; \hat{ }: p>0.05 vs. WT; @: p<0.05 vs. WT. SGSH activity is expressed as units/mg protein, 1 unit=nmol of 4MU released/17 hr. WT: wild-type mice. Liv: liver; Spl: spleen; Int: intestine; Hrt: heart; Kid: kidney; Mus: skeletal muscle.

FIG. 9 shows differential biodistribution in MPS IIIA mice following scAAV9-hSGSH^{op} gene delivery. MPS IIIA mice were treated at age 1-2 m with an IV injection of 5×10¹² vg/kg, 1×10¹³ vg/kg or 2×10¹³ vg/kg, or with combined IV (2×10¹² vg/kg) and IT injection (1×10¹² vg/kg). Tissues (n=4/group) were assayed in duplicate by qPCR at 1 m pi. Data are expressed as vector genome (vg) per diploid genomic DNA (dgDNA). *Vector genome was detected at <0.005×10⁵vg/ug gDNA in non-treated WT and MPS IIIA mice.

FIGS. 10A-10B show AAV9-mediated rapid and persistent rSGSH expression in the CNS and peripheral tissues in MPS IIIA mice following an IV vector delivery. MPS IIIA mice were treated at age 1-2 m with an IV injection of scAAV9-mCMV-hSGSH^{op} at 2e12 vg/kg, 8e12 vg/kg, 2e13 vg/kg, 4e13 vg/kg, or 8e13 vg/kg. Necropsy was performed at 1 m or 7 m pi for tissue analyses. FIG. 10A. Tissues (1 m pi) were assayed for SGSH activity: expressed as units/mg protein, 1 unit=nmol of 4MU released/17 hr. *There are <3% of normal SGSH activity in tissues in this MPS IIIA mouse model. FIG. 10B. Tissue sections (4 um) from mice treated with 2e13 vg/kg vector were assayed at 7 m pi by immunofluorescence for hSGSH and GFAP. Autofluorescence signals were also observed. IIIA: non-treated MPS IIIA mice; AAV: vector-treated MPS IIIA mice. Brain: CTX: cerebral cortex; TH: thalamus; ST: striatum; BS: brain stem; CB: cerebelum; G: granular layer; M: molecular layer; Closed arrows: rSGSH-positive cells; Notched arrows: rSGSH-positive blood vessel; Open arrows: myelinated nerve bundles; Arrowheads: Purkinje cells; Peripheral tissues: Liv: liver; Hrt: heart; Int: small intestine; ME: muscularis externa; SM: submucosa; Asterisks: peritoneal surface; Notched arrows: myenteric plexus neurons; Standard arrows: submocosal plexus neurons. Spl: spleen; RP: red pulp; WP: white pulp; Scale bar: 25 μm.

FIGS. 11A-11B show clearance of GAG accumulation in the CNS and periphery in MPS IIIA mice after an IV scAAV9-mCMV-hSGSH^{op} delivery. MPS IIIA mice were treated at age 1-2 m with an IV injection of scAAV9-mCMV-hSGSH^{op} at 2e12 vg/kg, 8e12 vg/kg, 2e13 vg/kg, 4e13 vg/kg, or 8e12 vg/kg. Necropsy was performed at 1 m or 7 m pi for tissue analyses. Controls were tissues from WT and non-treated MPS IIIA mice. FIG. 11A. Tissues (1 m pi) were assayed for GAG contents, as ug/mg wet tissue. *: p≤0.05 vs. IIIA; #: p>0.05 vs. IIIA; +: p≤0.05 vs WT; \hat{ }: p>0.05 vs. WT. FIG. 11B. Tissues sections (4 μm) from mice treated with 2e13 vg/kg vector were assayed at 7 m pi by immunofluorescence for LAMP1 and GFAP. Autofluorescence signals were also observed. IIIA: nontreated MPS IIIA mice; AAV: vector-treated MPS IIIA mice. Brain: CTX: cerebral cortex; TH: thalamus; ST: striatum; BS: brain stem; CB: cerebelum; G: granular layer; M: molecular layer; White outlines: Purkinje cells in between; Peripheral tissues: Liv: liver; Hrt: heart; Int: small intestine; ME: muscularis externa; SM: submucosa; Asterisks: peritoneal surface; Closed arrows: myenteric plexus neurons; Open arrows: submocosal plexus neurons. Spl: spleen; RP: red pulp; WP: white pulp; Scale bar: 25 μm.

FIGS. 12A-12C show correction of behavior deficits and extension of survival in MPS IIIA mice following an IV scAAV9-mCMV-hSGSH^{op} gene delivery. MPS IIIA mice were treated at age 1-2 m with an IV injection of scAAV9-mCMV-hSGSH^{op} at 2e12 vg/kg, 8e12 vg/kg, 2e13 vg/kg, 4e13 vg/kg, or 8e13 vg/kg. Controls were WT and non-treated MPS IIIA littermates. The animals were tested for behavior in a hidden task in Morris water maze at age 8 m (FIG. 12A, FIG. 12B). *: P<0.05 vs. IIIA; #: P.0.05 vs. IIIA; @: P<0.05 vs. WT; \hat{ }: P>0.05 vs. WT. Subsets of animals were observed for longevity (FIG. 12C). WT: wild-type littermates; IIIA: non-treated MPS IIIA mice.

FIG. 13 shows biodistribution of systemically delivered scAAV9-mCMV-hSGSH^{op} in MPS IIIA mice. MPS IIIA mice were treated at age 1 m with an IV injection of scAAV9-mCMV-hSGSH at 2e12 vg/kg, 8e12 vg/kg, 2e13 vg/kg, 4e13 vg/kg, or 8e13 vg/kg. Tissues were assayed in duplicate by qPCR at 1 m pi (n=4/group). Data expressed as 105 vector genome (vg) per μg diploid genomic DNA (dgDNA). *Vector genome was detected at <0.005×10⁵ vg/μg gDNA in non-treated WT and MPS IIIA mice.

### DETAILED DESCRIPTION OF THE INVENTION

The present invention will now be described with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, and other references mentioned herein are incorporated by reference in their entirety.

Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 CFR § 1.822 and established usage. See, e.g., PatentIn User Manual, 99-102 (November 1990) (U.S. Patent and Trademark Office).

Except as otherwise indicated, standard methods known to those skilled in the art may be used for the construction of recombinant parvovirus and AAV (rAAV) constructs, packaging vectors expressing the parvovirus Rep and/or Cap sequences, and transiently and stably transfected packaging cells. Such techniques are known to those skilled in the art. See, e.g., SAMBROOK et al. MOLECULAR CLONING: A LABORATORY MANUAL 4th Ed. (Cold Spring Harbor, NY, 2012); AUSUBEL et al. CURRENT PROTOCOLS IN MOLECULAR BIOLOGY (Green Publishing Associates, Inc. and John Wiley & Sons, Inc., New York).

Moreover, the present invention also contemplates that in some embodiments of the invention, any feature or combination of features set forth herein can be excluded or omitted.

To illustrate further, if, for example, the specification indicates that a particular amino acid can be selected from A, G, I, L and/or V, this language also indicates that the amino acid can be selected from any subset of these amino acid(s) for example A, G, I or L; A, G, I or V; A or G; only L; etc. as if each such subcombination is expressly set forth herein. Moreover, such language also indicates that one or more of the specified amino acids can be disclaimed. For example, in particular embodiments the amino acid is not A, G or I; is not A; is not G or V; etc. as if each such possible disclaimer is expressly set forth herein.

### Definitions

The following terms are used in the description herein and the appended claims.

The singular forms “a” and “an” are intended to include the plural forms as well, unless the context clearly indicates otherwise.

Furthermore, the term “about,” as used herein when referring to a measurable value such as an amount of the length of a polynucleotide or polypeptide sequence, dose, time, temperature, and the like, is meant to encompass variations of 10%, 5%, 1%, 0.5%, or even 0.1% of the specified amount.

Also as used herein, “and/or” refers to and encompasses any and all possible combinations of one or more of the associated listed items, as well as the lack of combinations when interpreted in the alternative (“or”).

As used herein, the transitional phrase “consisting essentially of” is to be interpreted as encompassing the recited materials or steps “and those that do not materially affect the basic and novel characteristic(s)” of the claimed invention (e.g., rAAV replication). Thus, the term “consisting essentially of” as used herein should not be interpreted as equivalent to “comprising.”

The term “consists essentially of” (and grammatical variants), as applied to a polynucleotide or polypeptide sequence of this invention, means a polynucleotide or polypeptide that consists of both the recited sequence (e.g., SEQ ID NO) and a total of ten or less (e.g., 1, 2, 3, 4, 5, 6, 7, 8, 9, or 10) additional nucleotides or amino acids on the 5′ and/or 3′ or N-terminal and/or C-terminal ends of the recited sequence such that the function of the polynucleotide or polypeptide is not materially altered. The total of ten or less additional nucleotides or amino acids includes the total number of additional nucleotides or amino acids on both ends added together. The term “materially altered,” as applied to polynucleotides of the invention, refers to an increase or decrease in ability to express the encoded polypeptide of at least about 50% or more as compared to the expression level of a polynucleotide consisting of the recited sequence. The term “materially altered,” as applied to polypeptides of the invention, refers to an increase or decrease in enzymatic activity of at least about 50% or more as compared to the activity of a polypeptide consisting of the recited sequence.

The term “parvovirus” as used herein encompasses the family Parvoviridae, including autonomously-replicating parvoviruses and dependoviruses. The autonomous parvoviruses include members of the genera Parvovirus, Erythrovirus, Densovirus, Iteravirus, and Contravirus. Exemplary autonomous parvoviruses include, but are not limited to, minute virus of mouse, bovine parvovirus, canine parvovirus, chicken parvovirus, feline panleukopenia virus, feline parvovirus, goose parvovirus, H1 parvovirus, muscovy duck parvovirus, snake parvovirus, and B19 virus. Other autonomous parvoviruses are known to those skilled in the art. See, e.g., FIELDS et al. VIROLOGY, volume 2, chapter 69 (4th ed., Lippincott-Raven Publishers).

The genus Dependovirus contains the adeno-associated viruses (AAV), including but not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, avian AAV, bovine AAV, canine AAV, goat AAV, snake AAV, equine AAV, and ovine AAV. See, e.g., FIELDS et al. VIROLOGY, volume 2, chapter 69 (4th ed., Lippincott-Raven Publishers); and Table 1.

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, snake AAV, avian AAV, bovine AAV, canine AAV, equine AAV, ovine AAV, goat AAV, shrimp AAV, and any other AAV now known or later discovered. See, e.g., FIELDS et al. VIROLOGY, volume 2, chapter 69 (4th ed., Lippincott-Raven Publishers). A number of relatively new AAV serotypes and clades have been identified (See, e.g., Gao et al. (2004) J. Virol. 78:6381; Moris et al. (2004) Virol. 33-: 375; and Table 1).

The parvovirus vectors, particles, and genomes of the present invention can be from, but are not limited to, AAV. The genomic sequences of various serotypes of AAV and the autonomous parvoviruses, as well as the sequences of the native ITRs, Rep proteins, and capsid subunits are known in the art. Such sequences may be found in the literature or in public databases such as GenBank. See, e.g., GenBank Accession Numbers NC_002077, NC_001401, NC_001729, NC_001863, NC_001829, NC_001862, NC_000883, NC_001701, NC_001510, NC_006152, NC_006261, AF063497, U89790, AF043303, AF028705, AF028704, J02275, J01901, J02275, X01457, AF288061, AH009962, AY028226, AY028223, AY631966, AX753250, EU285562, NC_001358, NC_001540, AF513851, AF513852 and AY530579; the disclosures of which are incorporated by reference herein for teaching parvovirus and AAV nucleic acid and amino acid sequences. See also, e.g., Bantel-Schaal et al. (1999) J. Virol. 73:939; Chiorini et al. (1997) J. Virol. 71:6823; Chiorini et al. (1999) J. Virol. 73:1309; Gao et al. (2002) Proc. Nat. Acad. Sci. USA 99:11854; Moris et al. (2004) Virol. 33-: 375-383; Mori et al. (2004) Virol. 330:375; Muramatsu et al. (1996) Virol. 221:208; Ruffing et al. (1994) J. Gen. Virol. 75:3385; Rutledge et al. (1998) J. Virol. 72:309; Schmidt et al. (2008) J. Virol. 82:8911; Shade et al. (1986) J. Virol. 58:921; Srivastava et al. (1983) J. Virol. 45:555; Xiao et al. (1999) J. Virol. 73:3994; international patent publications WO 00/28061, WO 99/61601, WO 98/11244; and U.S. Pat. No. 6,156,303; the disclosures of which are incorporated by reference herein for teaching parvovirus and AAV nucleic acid and amino acid sequences. See also Table 1. An early description of the AAV1, AAV2 and AAV3 ITR sequences is provided by Xiao, X., (1996), “Characterization of Adeno-associated virus (AAV) DNA replication and integration,” Ph.D. Dissertation, University of Pittsburgh, Pittsburgh, PA (incorporated herein in its entirety).

TABLE 1

| AAV | GenBank |
|---|---|
| Serotypes/Isolates | Accession Number |
| Clonal Isolates | |
| Avian AAV | AY186198, AY629583, |
| ATCC VR-865 | NC_004828 |
| Avian AAV | NC_006263, AY629583 |
| strain DA-1 | |
| Bovine AAV | NC_005889, AY388617 |
| AAV4 | NC_001829 |
| AAV5 | AY18065, AF085716 |
| Rh34 | AY243001 |
| Rh33 | AY243002 |
| Rh32 | AY243003 |
| AAV10 | AY631965 |
| AAV11 | AY631966 |
| AAV12 | DQ813647 |
| AAV13 | EU285562 |
| Clade A | |
| AAV1 | NC_002077, AF063497 |
| AAV6 | NC_001862 |
| Hu.48 | AY530611 |
| Hu 43 | AY530606 |
| Hu 44 | AY530607 |
| Hu 46 | AY530609 |
| Clade B | |
| Hu19 | AY530584 |
| Hu20 | AY530586 |
| Hu23 | AY530589 |
| Hu22 | AY530588 |
| Hu24 | AY530590 |
| Hu21 | AY530587 |
| Hu27 | AY530592 |
| Hu28 | AY530593 |
| Hu29 | AY530594 |
| Hu63 | AY530624 |
| Hu64 | AY530625 |
| Hu13 | AY530578 |
| Hu56 | AY530618 |
| Hu57 | AY530619 |
| Hu49 | AY530612 |
| Hu58 | AY530620 |
| Hu34 | AY530598 |
| Hu35 | AY530599 |
| AAV2 | NC_001401 |
| Hu45 | AY530608 |
| Hu47 | AY530610 |
| Hu51 | AY530613 |
| Hu52 | AY530614 |
| Hu T41 | AY695378 |
| Hu S17 | AY695376 |
| Hu T88 | AY695375 |
| Hu T71 | AY695374 |
| Hu T70 | AY695373 |
| Hu T40 | AY695372 |
| Hu T32 | AY695371 |
| Hu T17 | AY695370 |
| Hu LG15 | AY695377 |
| Clade C | |
| AAV 3 | NC_001729 |
| AAV 3B | NC_001863 |
| Hu9 | AY530629 |
| Hu10 | AY530576 |
| Hu11 | AY530577 |
| Hu53 | AY530615 |
| Hu55 | AY530617 |
| Hu54 | AY530616 |
| Hu7 | AY530628 |
| Hu18 | AY530583 |
| Hu15 | AY530580 |
| Hu16 | AY530581 |
| Hu25 | AY530591 |
| Hu60 | AY530622 |
| Ch5 | AY243021 |
| Hu3 | AY530595 |
| Hu1 | AY530575 |
| Hu4 | AY530602 |
| Hu2 | AY530585 |
| Hu61 | AY530623 |
| Clade D | |
| Rh62 | AY530573 |
| Rh48 | AY530561 |
| Rh54 | AY530567 |
| Rh55 | AY530568 |
| Cy2 | AY243020 |
| AAV7 | AF513851 |
| Rh35 | AY243000 |
| Rh37 | AY242998 |
| Rh36 | AY242999 |
| Cy6 | AY243016 |
| Cy4 | AY243018 |
| Cy3 | AY243019 |
| Cy5 | AY243017 |
| Rh13 | AY243013 |
| Clade E | |
| Rh38 | AY530558 |
| Hu66 | AY530626 |
| Hu42 | AY530605 |
| Hu67 | AY530627 |
| Hu40 | AY530603 |
| Hu41 | AY530604 |
| Hu37 | AY530600 |
| Rh40 | AY530559 |
| Rh2 | AY243007 |
| Bb1 | AY243023 |
| Bb2 | AY243022 |
| Rh10 | AY243015 |
| Hu17 | AY530582 |
| Hu6 | AY530621 |
| Rh25 | AY530557 |
| Pi2 | AY530554 |
| Pi1 | AY530553 |
| Pi3 | AY530555 |
| Rh57 | AY530569 |
| Rh50 | AY530563 |
| Rh49 | AY530562 |
| Hu39 | AY530601 |
| Rh58 | AY530570 |
| Rh61 | AY530572 |
| Rh52 | AY530565 |
| Rh53 | AY530566 |
| Rh51 | AY530564 |
| Rh64 | AY530574 |
| Rh43 | AY530560 |
| AAV8 | AF513852 |
| Rh8 | AY242997 |
| Rh1 | AY530556 |
| Clade F | |
| AAV9 (Hu14) | AY530579 |
| Hu31 | AY530596 |
| Hu32 | AY530597 |

The term “tropism” as used herein refers to entry of the virus into the cell, optionally and preferably followed by expression (e.g., transcription and, optionally, translation) of sequences carried by the viral genome in the cell, e.g., for a recombinant virus, expression of the heterologous nucleotide sequences(s). Those skilled in the art will appreciate that transcription of a heterologous nucleic acid sequence from the viral genome may not be initiated in the absence of trans-acting factors, e.g., for an inducible promoter or otherwise regulated nucleic acid sequence. In the case of AAV, gene expression from the viral genome may be from a stably integrated provirus, from a non-integrated episome, as well as any other form in which the virus may take within the cell.

As used herein, “transduction” of a cell by parvovirus or AAV refers to parvovirus/AAV-mediated transfer of genetic material into the cell. See, e.g., FIELDS et al. VIROLOGY, volume 2, chapter 69 (3d ed., Lippincott-Raven Publishers).

The terms “5′ portion” and “3′ portion” are relative terms to define a spatial relationship between two or more elements. Thus, for example, a “3′ portion” of a polynucleotide indicates a segment of the polynucleotide that is downstream of another segment. The term “3′ portion” is not intended to indicate that the segment is necessarily at the 3′ end of the polynucleotide, or even that it is necessarily in the 3′ half of the polynucleotide, although it may be. Likewise, a “5′ portion” of a polynucleotide indicates a segment of the polynucleotide that is upstream of another segment. The term “5′ portion” is not intended to indicate that the segment is necessarily at the 5′ end of the polynucleotide, or even that it is necessarily in the 5′ half of the polynucleotide, although it may be.

As used herein, the term “polypeptide” encompasses both peptides and proteins, unless indicated otherwise.

A “polynucleotide” is a sequence of nucleotide bases, and may be RNA, DNA or DNA-RNA hybrid sequences (including both naturally occurring and non-naturally occurring nucleotides), and can be either single or double stranded DNA sequences.

The term “sequence identity,” as used herein, has the standard meaning in the art. As is known in the art, a number of different programs can be used to identify whether a polynucleotide or polypeptide has sequence identity or similarity to a known sequence. Sequence identity or similarity may be determined using standard techniques known in the art, including, but not limited to, the local sequence identity algorithm of Smith & Waterman (1981) Adv. Appl. Math. 2:482, by the sequence identity alignment algorithm of Needleman & Wunsch (1970) J. Mol. Biol. 48:443, by the search for similarity method of Pearson & Lipman (1988) Proc. Natl. Acad. Sci. USA 85:2444, by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al. (1984) Nucl. Acid Res. 12:387, preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle (1987), J. Mol. Evol. 35:351; the method is similar to that described by Higgins & Sharp (1989) CABIOS 5:151.

Another example of a useful algorithm is the BLAST algorithm, described in Altschul et al. (1990) J. Mol. Biol. 215:403 and Karlin et al. (1993) Proc. Natl. Acad. Sci. USA 90:5873. A particularly useful BLAST program is the WU-BLAST-2 program which was obtained from Altschul et al. (1996) Meth. Enzymol. 266:460. WU-BLAST-2 uses several search parameters, which are preferably set to the default values. The parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al. (1997) Nucleic Acids Res. 25:3389.

A percentage amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

In a similar manner, percent nucleic acid sequence identity is defined as the percentage of nucleotide residues in the candidate sequence that are identical with the nucleotides in the polynucleotide specifically disclosed herein.

The alignment may include the introduction of gaps in the sequences to be aligned. In addition, for sequences which contain either more or fewer nucleotides than the polynucleotides specifically disclosed herein, it is understood that in one embodiment, the percentage of sequence identity will be determined based on the number of identical nucleotides in relation to the total number of nucleotides. Thus, for example, sequence identity of sequences shorter than a sequence specifically disclosed herein, will be determined using the number of nucleotides in the shorter sequence, in one embodiment. In percent identity calculations relative weight is not assigned to various manifestations of sequence variation, such as insertions, deletions, substitutions, etc.

In one embodiment, only identities are scored positively (+1) and all forms of sequence variation including gaps are assigned a value of “0,” which obviates the need for a weighted scale or parameters as described below for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

As used herein, an “isolated” polynucleotide (e.g., an “isolated DNA” or an “isolated RNA”) means a polynucleotide separated or substantially free from at least some of the other components of the naturally occurring organism or virus, for example, the cell or viral structural components or other polypeptides or nucleic acids commonly found associated with the polynucleotide.

Likewise, an “isolated” polypeptide means a polypeptide that is separated or substantially free from at least some of the other components of the naturally occurring organism or virus, for example, the cell or viral structural components or other polypeptides or nucleic acids commonly found associated with the polypeptide.

A “therapeutic polypeptide” is a polypeptide that may alleviate or reduce symptoms that result from an absence or defect in a protein in a cell or subject. Alternatively, a “therapeutic polypeptide” is one that otherwise confers a benefit to a subject, e.g., anti-cancer effects or improvement in transplant survivability.

As used herein, the term “modified,” as applied to a polynucleotide or polypeptide sequence, refers to a sequence that differs from a wild-type sequence due to one or more deletions, additions, substitutions, or any combination thereof.

As used herein, by “isolate” or “purify” (or grammatical equivalents) a virus vector, it is meant that the virus vector is at least partially separated from at least some of the other components in the starting material.

By the terms “treat,” “treating,” or “treatment of” (and grammatical variations thereof) it is meant that the severity of the subject's condition is reduced, at least partially improved or stabilized and/or that some alleviation, mitigation, decrease or stabilization in at least one clinical symptom is achieved and/or there is a delay in the progression of the disease or disorder.

The terms “prevent,” “preventing,” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the present invention.

A “treatment effective” amount as used herein is an amount that is sufficient to provide some improvement or benefit to the subject. Alternatively stated, a “treatment effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

The terms “heterologous nucleotide sequence” and “heterologous nucleic acid” are used interchangeably herein and refer to a sequence that is not naturally occurring in the virus. In some embodiments, the heterologous nucleic acid comprises an open reading frame that encodes a polypeptide (e.g., for delivery to a cell or subject).

The term “operably linked” refers to the functional relation and the location of an expression control sequence (e.g., promoter, terminator, poly (A) signal, etc.) with respect to the coding sequence of interest (e.g., a promoter or enhancer is operably linked to a coding sequence if it affects the transcription of the sequence). Generally, a promoter operably linked is contiguous to the sequence of interest.

As used herein, the terms “virus vector,” “vector” or “gene delivery vector” refer to a virus (e.g., AAV) particle that functions as a nucleic acid delivery vehicle, and which comprises the vector genome (e.g., viral DNA [vDNA]) packaged within a virion. Alternatively, in some contexts, the term “vector” may be used to refer to the vector genome/vDNA alone or a plasmid.

The virus vectors of the invention can further be duplexed parvovirus particles as described in international patent publication WO 01/92551 (the disclosure of which is incorporated herein by reference in its entirety). Thus, in some embodiments, double stranded (duplex) genomes can be packaged.

An “AAV vector genome,” “rAAV vector genome” or “rAAV genome” is an AAV genome (i.e., vDNA) that comprises one or more heterologous nucleic acid sequences. rAAV vectors generally require only the 145 base ITR in cis to generate virus. All other viral sequences are dispensable and may be supplied in trans (Muzyczka (1992) Curr. Topics Microbiol. Immunol. 158:97). Typically, the rAAV vector genome will only retain the one or more ITR sequence so as to maximize the size of the transgene that can be efficiently packaged by the vector. The structural and non-structural protein coding sequences may be provided in trans (e.g., from a vector, such as a plasmid, or by stably integrating the sequences into a packaging cell). In embodiments of the invention the rAAV vector genome comprises at least one ITR sequence (e.g., AAV ITR sequence), optionally two ITRs (e.g., two AAV ITRs), which typically will be at the 5′ and 3′ ends of the vector genome and flank the heterologous nucleic acid, but need not be contiguous thereto. The ITRs can be the same or different from each other.

The term “terminal repeat” or “TR” includes any viral terminal repeat or synthetic sequence that forms a hairpin structure and functions as an inverted terminal repeat (i.e., mediates the desired functions such as replication, virus packaging, integration and/or provirus rescue, and the like). The ITR can be an AAV ITR or a non-AAV ITR. For example, a non-AAV ITR sequence such as those of other parvoviruses (e.g., canine parvovirus, bovine parvovirus, mouse parvovirus, porcine parvovirus, human parvovirus B-19) or the SV40 hairpin that serves as the origin of SV40 replication can be used as an ITR, which can further be modified by truncation, substitution, deletion, insertion and/or addition. Further, the ITR can be partially or completely synthetic, such as the “double-D sequence” as described in U.S. Pat. No. 5,478,745 to Samulski et al.

Parvovirus genomes have palindromic sequences at both their 5′ and 3′ ends. The palindromic nature of the sequences leads to the formation of a hairpin structure that is stabilized by the formation of hydrogen bonds between the complementary base pairs. This hairpin structure is believed to adopt a “Y” or a “T” shape. See, e.g., FIELDS et al. VIROLOGY, volume 2, chapters 69 & 70 (4th ed., Lippincott-Raven Publishers).

An “AAV inverted terminal repeat” or “AAV ITR” may be from any AAV, including but not limited to serotypes 1, 2, 3a, 3b, 4, 5, 6, 7, 8, 9, 10, 11, or 13, snake AAV, avian AAV, bovine AAV, canine AAV, equine AAV, ovine AAV, goat AAV, shrimp AAV, or any other AAV now known or later discovered (see, e.g., Table 1). An AAV ITR need not have the native terminal repeat sequence (e.g., a native AAV ITR sequence may be altered by insertion, deletion, truncation and/or missense mutations), as long as the terminal repeat mediates the desired functions, e.g., replication, virus packaging, persistence, and/or provirus rescue, and the like. In some embodiments, at least one AAV ITR is an AAV2 ITR. In some embodiments, at least one AAV ITR is an AAV2 ITR with deletion of terminal resolution site to force generation of self-complementary dimeric genomes (dTR). In some embodiments, a wild-type AAV2 inverted terminal repeat and an AAV2 inverted terminal repeat with deletion of the terminal resolution site are used.

The virus vectors of the invention can further be “targeted” virus vectors (e.g., having a directed tropism) and/or a “hybrid” parvovirus (i.e., in which the viral ITRs and viral capsid are from different parvoviruses) as described in international patent publication WO 00/28004 and Chao et al. (2000) Mol. Therapy 2:619.

Further, the viral capsid or genomic elements can contain other modifications, including insertions, deletions and/or substitutions.

The term “template” or “substrate” is used herein to refer to a polynucleotide sequence that may be replicated to produce the parvovirus viral DNA. For the purpose of vector production, the template will typically be embedded within a larger nucleotide sequence or construct, including but not limited to a plasmid, naked DNA vector, bacterial artificial chromosome (BAC), yeast artificial chromosome (YAC) or a viral vector (e.g., adenovirus, herpesvirus, Epstein-Barr Virus, AAV, baculoviral, retroviral vectors, and the like). Alternatively, the template may be stably incorporated into the chromosome of a packaging cell.

As used herein, parvovirus or AAV “Rep coding sequences” indicate the nucleic acid sequences that encode the parvoviral or AAV non-structural proteins that mediate viral replication and the production of new virus particles. The parvovirus and AAV replication genes and proteins have been described in, e.g., FIELDS et al. VIROLOGY, volume 2, chapters 69 & 70 (4th ed., Lippincott-Raven Publishers).

The “Rep coding sequences” need not encode all of the parvoviral or AAV Rep proteins. For example, with respect to AAV, the Rep coding sequences do not need to encode all four AAV Rep proteins (Rep78, Rep 68, Rep52 and Rep40), in fact, it is believed that AAV5 only expresses the spliced Rep68 and Rep40 proteins. In representative embodiments, the Rep coding sequences encode at least those replication proteins that are necessary for viral genome replication and packaging into new virions. The Rep coding sequences will generally encode at least one large Rep protein (i.e., Rep78/68) and one small Rep protein (i.e., Rep52/40). In particular embodiments, the Rep coding sequences encode the AAV Rep78 protein and the AAV Rep52 and/or Rep40 proteins. In other embodiments, the Rep coding sequences encode the Rep68 and the Rep52 and/or Rep40 proteins. In a still further embodiment, the Rep coding sequences encode the Rep68 and Rep52 proteins, Rep68 and Rep40 proteins, Rep78 and Rep52 proteins, or Rep78 and Rep40 proteins.

As used herein, the term “large Rep protein” refers to Rep68 and/or Rep78. Large Rep proteins of the claimed invention may be either wild-type or synthetic. A wild-type large Rep protein may be from any parvovirus or AAV, including but not limited to serotypes 1, 2, 3a, 3b, 4, 5, 6, 7, 8, 9, 10, 11, or 13, or any other AAV now known or later discovered (see, e.g., Table 1). A synthetic large Rep protein may be altered by insertion, deletion, truncation and/or missense mutations.

Those skilled in the art will further appreciate that it is not necessary that the replication proteins be encoded by the same polynucleotide. For example, for MVM, the NS-1 and NS-2 proteins (which are splice variants) may be expressed independently of one another. Likewise, for AAV, the p19 promoter may be inactivated and the large Rep protein(s) expressed from one polynucleotide and the small Rep protein(s) expressed from a different polynucleotide. Typically, however, it will be more convenient to express the replication proteins from a single construct. In some systems, the viral promoters (e.g., AAV p19 promoter) may not be recognized by the cell, and it is therefore necessary to express the large and small Rep proteins from separate expression cassettes. In other instances, it may be desirable to express the large Rep and small Rep proteins separately, i.e., under the control of separate transcriptional and/or translational control elements. For example, it may be desirable to control expression of the large Rep proteins, so as to decrease the ratio of large to small Rep proteins. In the case of insect cells, it may be advantageous to down-regulate expression of the large Rep proteins (e.g., Rep78/68) to avoid toxicity to the cells (see, e.g., Urabe et al. (2002) Human Gene Therapy 13:1935).

As used herein, the parvovirus or AAV “cap coding sequences” encode the structural proteins that form a functional parvovirus or AAV capsid (i.e., can package DNA and infect target cells). Typically, the cap coding sequences will encode all of the parvovirus or AAV capsid subunits, but less than all of the capsid subunits may be encoded as long as a functional capsid is produced. Typically, but not necessarily, the cap coding sequences will be present on a single nucleic acid molecule.

The capsid structure of autonomous parvoviruses and AAV are described in more detail in BERNARD N. FIELDS et al. VIROLOGY, volume 2, chapters 69 & 70 (4th ed., Lippincott-Raven Publishers).

### Vectors Expressing SGSH

The present invention provides vectors, e.g., virus vectors, e.g., parvovirus vectors, e.g., AAV vectors, that comprise a nucleotide sequence encoding SGSH that is codon-optimized for expression in human cells and are capable of providing both enhanced expression and enhanced secretion of SGSH from cells infected with the vector.

One aspect of the invention relates to a recombinant nucleic acid comprising, consisting essentially of, or consisting of a nucleotide sequence encoding human SGSH that is codon-optimized for expression in human cells. In certain embodiments, the nucleic acid is a non-naturally occurring sequence. In some embodiments, the nucleic acid comprises, consists essentially of, or consists of a nucleotide sequence that is at least 90% identical to SEQ ID NO:1, e.g., at least 91%, 92%, 93%, 94%, 95%, 96%, 97%, 98%, or 99% identical to SEQ ID NO:1. In some embodiments, the nucleic acid comprises, consists essentially of, or consists of a nucleotide sequence that is at least 99% identical to SEQ ID NO:1. In some embodiments, the nucleic acid comprises, consists essentially of, or consists of the nucleotide sequence of SEQ ID NO:1. In some embodiments, the nucleic acid comprises at least 10 contiguous nucleotides of SEQ ID NO:1, e.g., at least 10, 25, 50, 100, 200, 300, 400, 500, 600, 700, 800, 900, 1000, 1100, 1200, 1300, 1400, 1500, or more.

Methods of codon optimizing a nucleotide sequence to maximize expression in an organism are known in the art and can be carried out using software available to the public. The wild-type sequence of human SGSH is known in the art and shown in SEQ ID NO:2.

In some embodiments, codon optimization of the 60 nucleotides encoding the amino terminal 20 amino acid residues of human SGSH (SEQ ID NO:16) is unexpectedly sufficient to enhance both expression and secretion of human SGSH from cells, whereas codon optimization throughout the coding sequence (see SEQ ID NOs: 8-15) either fails to enhance SGSH expression or decreases expression to <80% of the wild-type vector. Thus, in some embodiments, the nucleic acid encoding human SGSH comprises a nucleotide sequence wherein the 60 nucleotides at the 5′ end have been codon optimized and the remaining 1449 nucleotides at the 3′ end are 100% identical to the wild-type human SGSH nucleic acid sequence. In some embodiments, the sequence of the 60 nucleotides at the 5′ end of the codon optimized human SGSH nucleic acid share between 70% and 99% sequence identity with the sequence of the 60 nucleotides at the 5′ end of the wild-type human SGSH nucleic acid. In some embodiments, the nucleic acid encoding human SGSH comprises a nucleotide sequence that is at least 75% identical to SEQ ID NO:16, e.g., 76%, 77%, 78%, 79%, 80%, 815, 82%, 83%, 84%, 85%, 86%, 87%, 88%, 89%, 90%, 91%, 92%, 93%, 94%, 95%, 96%, 97%, 98%, or 99% identical to SEQ ID NO:16. In some embodiments, the codon-optimized nucleic acid encoding human SGSH comprises a sequence, wherein at least 5 out of 20 of the 5′ codons have been codon optimized, e.g., 6, 7, 8, 9, 10, 11, 12, 13 or 14 of the 20 5′ codons have been codon optimized relative to SEQ ID NO:2.

The invention also provides a viral vector genome comprising the SGSH nucleic acid of the invention. The viral vector genome may be a parvovirus vector genome, e.g., an AAV vector genome. In some embodiments, the AAV vector genome is a self-complementary AAV vector genome. The viral vector genome may further comprise a promoter operably linked to the SGSH nucleic acid. In some embodiments, the promoter may be a constitutive promoter, e.g., the CBA promoter or the human elongation factor 1 alpha (EF1a) promoter. In other embodiments, the promoter may be a tissue-specific or preferred promoter. In some embodiments, the promoter is the miniature cytomegalovirus (mCMV) promoter. The mCMV promoter may comprise the sequence of SEQ ID NO:3 or a sequence at least 90% identical thereto, e.g., at least 91%, 92%, 93%, 94%, 95%, 96%, 97%, 98%, or 99% identical thereto. The viral vector genome may further comprise a polyadenylation signal operably linked to the SGSH nucleic acid. In some embodiments, the polyadenylation signal is the SV40 polyadenylation signal, which may comprise the sequence of SEQ ID NO: 4 or a sequence at least 90% identical thereto, e.g., at least 91%, 92%, 93%, 94%, 95%, 96%, 97%, 98%, or 99% identical thereto. In some embodiments, the AAV vector genome comprises, consists essentially of, or consists of a nucleotide sequence that is at least 90% identical to SEQ ID NO:5, e.g., at least 91%, 92%, 93%, 94%, 95%, 96%, 97%, 98%, or 99% identical to SEQ ID NO:5.

The invention further provides a vector comprising the viral vector genome of the invention. In some embodiments, the vector is a plasmid. In some embodiments, the vector comprises the nucleotide sequence of SEQ ID NO:7 or a sequence at least 90% identical thereto, e.g., at least 91%, 92%, 93%, 94%, 95%, 96%, 97%, 98%, or 99% identical thereto

The invention further provides a cell in vitro comprising the AAV vector genome of the invention, e.g., stably incorporated into the genome of the cell. In some embodiments, the cell is a human cell. In some embodiments, the cell is not a germ cell or stem cell. The invention further provides a recombinant virus particle (e.g., a recombinant parvovirus particle, e.g., an AAV particle, e.g., an AAV9 particle) comprising the viral vector genome of the invention. Viral vectors and viral particles are discussed further below.

### Methods of Producing Virus Vectors

The present invention further provides methods of producing virus vectors. In one embodiment, the present invention provides a method of producing a recombinant parvovirus particle (e.g., an AAV particle), comprising providing to a cell permissive for parvovirus replication: (a) a recombinant parvovirus template comprising (i) the nucleic acid encoding SGSH of the invention, and (ii) a parvovirus ITR; (b) a polynucleotide comprising Rep and Cap coding sequences; under conditions sufficient for the replication and packaging of the recombinant parvovirus template; whereby recombinant parvovirus particles are produced in the cell. In a particular embodiment, the present invention provides a method of producing a recombinant AAV particle comprising an AAV capsid by providing a cell in vitro with nucleic acids comprising AAV Cap and AAV Rep coding sequences and helper functions for generating a productive AAV infection and allowing assembly of the recombinant AAV particle comprising the AAV capsid and encapsidating the AAV vector genome. Conditions sufficient for the replication and packaging of the recombinant parvovirus template can be, e.g., the presence of AAV sequences sufficient for replication of the parvovirus template and encapsidation into parvovirus capsids (e.g., parvovirus rep sequences and parvovirus cap sequences) and helper sequences from adenovirus and/or herpesvirus. In particular embodiments, the parvovirus template comprises two parvovirus ITR sequences, which are located 5′ and 3′ to the heterologous nucleic acid sequence, although they need not be directly contiguous thereto.

In some embodiments, the recombinant parvovirus template comprises an ITR that is not resolved by Rep to make duplexed AAV vectors as described in international patent publication WO 01/92551.

The parvovirus template and parvovirus rep and cap sequences are provided under conditions such that virus vector comprising the parvovirus template packaged within the parvovirus capsid is produced in the cell. The method can further comprise the step of collecting the virus vector from the cell. The virus vector can be collected from the medium and/or by lysing the cells.

The cell can be a cell that is permissive for parvoviral viral replication. Any suitable cell known in the art may be employed. In particular embodiments, the cell is a mammalian cell (e.g., a primate or human cell). As another option, the cell can be a trans-complementing packaging cell line that provide functions deleted from a replication-defective helper virus, e.g., 293 cells or other Ela trans-complementing cells.

The parvovirus replication and capsid sequences may be provided by any method known in the art. Current protocols typically express the parvovirus rep/cap genes on a single plasmid. The parvovirus replication and packaging sequences need not be provided together, although it may be convenient to do so. The parvovirus rep and/or cap sequences may be provided by any viral or non-viral vector. For example, the rep/cap sequences may be provided by a hybrid adenovirus or herpesvirus vector (e.g., inserted into the Ela or E3 regions of a deleted adenovirus vector). EBV vectors may also be employed to express the parvovirus cap and rep genes. One advantage of this method is that EBV vectors are episomal, yet will maintain a high copy number throughout successive cell divisions (i.e., are stably integrated into the cell as extra-chromosomal elements, designated as an “EBV based nuclear episome,” see Margolski, (1992) Curr. Top. Microbiol. Immun. 158:67).

As a further alternative, the rep/cap sequences may be stably incorporated into a cell.

Typically the parvovirus rep/cap sequences will not be flanked by the TRs, to prevent rescue and/or packaging of these sequences.

The parvovirus template can be provided to the cell using any method known in the art. For example, the template can be supplied by a non-viral (e.g., plasmid) or viral vector. In particular embodiments, the parvovirus template is supplied by a herpesvirus or adenovirus vector (e.g., inserted into the Ela or E3 regions of a deleted adenovirus). As another illustration, Palombo et al. (1998) J. Virology 72:5025, describes a baculovirus vector carrying a reporter gene flanked by the AAV TRs. EBV vectors may also be employed to deliver the template, as described above with respect to the rep/cap genes.

In another representative embodiment, the parvovirus template is provided by a replicating rAAV virus. In still other embodiments, an AAV provirus comprising the parvovirus template is stably integrated into the chromosome of the cell. In still further embodiments, the AAV particle produced is an AAV9 particle.

To enhance virus titers, helper virus functions (e.g., adenovirus or herpesvirus) that promote a productive parvovirus infection can be provided to the cell. Helper virus sequences necessary for parvovirus replication are known in the art. Typically, these sequences will be provided by a helper adenovirus or herpesvirus vector. Alternatively, the adenovirus or herpesvirus sequences can be provided by another non-viral or viral vector, e.g., as a non-infectious adenovirus miniplasmid that carries all of the helper genes that promote efficient parvovirus production as described by Ferrari et al. (1997) Nature Med. 3:1295, and U.S. Pat. Nos. 6,040,183 and 6,093,570.

Further, the helper virus functions may be provided by a packaging cell with the helper sequences embedded in the chromosome or maintained as a stable extrachromosomal element. Generally, the helper virus sequences cannot be packaged into AAV virions, e.g., are not flanked by ITRs.

Those skilled in the art will appreciate that it may be advantageous to provide the parvovirus replication and capsid sequences and the helper virus sequences (e.g., adenovirus sequences) on a single helper construct. This helper construct may be a non-viral or viral construct. As one nonlimiting illustration, the helper construct can be a hybrid adenovirus or hybrid herpesvirus comprising the AAV rep/cap genes.

In one particular embodiment, the parvovirus rep/cap sequences and the adenovirus helper sequences are supplied by a single adenovirus helper vector. This vector can further comprise the parvovirus template. The parvovirus rep/cap sequences and/or the parvovirus template can be inserted into a deleted region (e.g., the Ela or E3 regions) of the adenovirus.

In a further embodiment, the parvovirus rep/cap sequences and the adenovirus helper sequences are supplied by a single adenovirus helper vector. According to this embodiment, the parvovirus template can be provided as a plasmid template.

In another illustrative embodiment, the parvovirus rep/cap sequences and adenovirus helper sequences are provided by a single adenovirus helper vector, and the parvovirus template is integrated into the cell as a provirus. Alternatively, the parvovirus template is provided by an EBV vector that is maintained within the cell as an extrachromosomal element (e.g., as an EBV based nuclear episome).

In a further exemplary embodiment, the parvovirus rep/cap sequences and adenovirus helper sequences are provided by a single adenovirus helper. The parvovirus template can be provided as a separate replicating viral vector. For example, the parvovirus template can be provided by a parvovirus particle or a second recombinant adenovirus particle.

According to the foregoing methods, the hybrid adenovirus vector typically comprises the adenovirus 5′ and 3′ cis sequences sufficient for adenovirus replication and packaging (i.e., the adenovirus terminal repeats and PAC sequence). The parvovirus rep/cap sequences and, if present, the AAV template are embedded in the adenovirus backbone and are flanked by the 5′ and 3′ cis sequences, so that these sequences may be packaged into adenovirus capsids. As described above, the adenovirus helper sequences and the parvovirus rep/cap sequences are generally not flanked by ITRs so that these sequences are not packaged into the parvovirus virions.

Zhang et al. ((2001) Gene Ther. 18:704-12) describe a chimeric helper comprising both adenovirus and the AAV rep and cap genes.

Herpesvirus may also be used as a helper virus in parvovirus packaging methods. Hybrid herpesviruses encoding the parvovirus Rep protein(s) may advantageously facilitate scalable parvovirus vector production schemes. A hybrid herpes simplex virus type I (HSV-1) vector expressing the AAV-2 rep and cap genes has been described (Conway et al. (1999) Gene Ther. 6:986 and WO 00/17377.

As a further alternative, the virus vectors of the invention can be produced in insect cells using baculovirus vectors to deliver the rep/cap genes and parvovirus template as described, for example, by Urabe et al. (2002) Human Gene Ther. 13:1935-43.

Parvovirus vector stocks free of contaminating helper virus may be obtained by any method known in the art. For example, parvovirus and helper virus may be readily differentiated based on size. Parvovirus may also be separated away from helper virus based on affinity for a heparin substrate (Zolotukhin et al. (1999) Gene Therapy 6:973). Deleted replication-defective helper viruses can be used so that any contaminating helper virus is not replication competent. As a further alternative, an adenovirus helper lacking late gene expression may be employed, as only adenovirus early gene expression is required to mediate packaging of parvovirus. Adenovirus mutants defective for late gene expression are known in the art (e.g., ts100K and ts149 adenovirus mutants).

### Recombinant Virus Vectors

The virus vectors of the present invention are useful for the delivery of nucleic acids to cells in vitro, ex vivo, and in vivo. In particular, the virus vectors can be advantageously employed to deliver or transfer nucleic acids to animal, including mammalian, cells. In particular, the virus vectors of the present invention are useful for the delivery of a nucleic acid encoding SGSH to a subject.

It will be understood by those skilled in the art that the nucleic acid encoding SGSH can be operably linked with appropriate control sequences. For example, the nucleic acid can be operably linked with expression control elements, such as transcription/translation control signals, origins of replication, polyadenylation signals, internal ribosome entry sites (IRES), promoters, and/or enhancers, and the like.

Those skilled in the art will appreciate that a variety of promoter/enhancer elements can be used depending on the level and tissue-specific expression desired. The promoter/enhancer can be constitutive or inducible, depending on the pattern of expression desired. The promoter/enhancer can be native or foreign and can be a natural or a synthetic sequence. By foreign, it is intended that the transcriptional initiation region is not found in the wild-type host into which the transcriptional initiation region is introduced.

In particular embodiments, the promoter/enhancer elements can be native to the target cell or subject to be treated. In representative embodiments, the promoters/enhancer element can be native to the SGSH nucleic acid sequence. The promoter/enhancer element is generally chosen so that it functions in the target cell(s) of interest. Further, in particular embodiments the promoter/enhancer element is a mammalian promoter/enhancer element. In particular embodiments, the promoter/enhancer element functions in all cells so that SGSH is expressed systemically. The promoter/enhancer element may be constitutive or inducible.

Inducible expression control elements are typically advantageous in those applications in which it is desirable to provide regulation over expression of the nucleic acid sequence. Inducible promoters/enhancer elements for gene delivery can be tissue-specific or tissue-preferred promoter/enhancer elements, and include neuron-specific or neuron-preferred promoter/enhancer elements. Other inducible promoter/enhancer elements include hormone-inducible and metal-inducible elements. Exemplary inducible promoters/enhancer elements include, but are not limited to, a Tet on/off element, a RU486-inducible promoter, an ecdysone-inducible promoter, a rapamycin-inducible promoter, and a metallothionein promoter.

In embodiments wherein the nucleic acid sequence is transcribed and then translated in the target cells, specific initiation signals are generally included for efficient translation of inserted protein coding sequences. These exogenous translational control sequences, which may include the ATG initiation codon and adjacent sequences, can be of a variety of origins, both natural and synthetic.

The virus vectors of the invention can be parvovirus vectors, e.g., AAV vectors. The AAV vectors may be any AAV serotype. In some embodiments, the AAV vector is an AAV2, AAV8, or AAV9 vector. In some embodiments, the AAV vector is a hybrid vector, e.g., one having a capsid protein from one serotype and a genome from another serotype or one having a synthetic capsid protein. In certain embodiments, the vector comprises a hybrid capsid with an altered tropism. In one example the hybrid capsid comprising a glycan binding site (e.g., a galactose binding site) from one serotype (e.g., AAV9) in a capsid sequence from another serotype (e.g., AAV8) (see, e.g., WO 2014/144229, incorporated by reference herein in its entirety).

The virus vectors according to the present invention provide a means for delivering SGSH nucleic acids (codon optimized SGSH nucleic acids) into a broad range of cells, including dividing and non-dividing cells. The virus vectors can be employed to deliver the nucleic acid to a cell in vitro, e.g., to produce a polypeptide in vitro or for ex vivo gene therapy. The virus vectors are additionally useful in a method of delivering the nucleic acid to a subject in need thereof, e.g., to express SGSH. In this manner, the polypeptide can be produced in vivo in the subject. The subject can be in need of the polypeptide because the subject has a deficiency of the polypeptide. Further, the method can be practiced because the production of the polypeptide in the subject may impart some beneficial effect.

The virus vectors can also be used to produce SGSH in cultured cells or in a subject (e.g., using the subject as a bioreactor to produce the polypeptide or to observe the effects of the polypeptide on the subject, for example, in connection with screening methods).

The virus vectors of the present invention can be employed to deliver a nucleic acid encoding SGSH to treat and/or prevent any disease state for which it is beneficial to deliver SGSH, e.g., MPS IIIA.

Virus vectors according to the instant invention find use in diagnostic and screening methods, whereby the SGSH nucleic acid is transiently or stably expressed in a cell culture system, in an organ or organ culture, or alternatively, a transgenic animal model.

The virus vectors of the present invention can also be used for various non-therapeutic purposes, including but not limited to use in protocols to assess gene targeting, clearance, transcription, translation, etc., as would be apparent to one skilled in the art. The virus vectors can also be used for the purpose of evaluating safety (spread, toxicity, immunogenicity, etc.). Such data, for example, are considered by the United States Food and Drug Administration as part of the regulatory approval process prior to evaluation of clinical efficacy.

Alternatively, the virus vector may be administered to a cell ex vivo, and the altered cell is administered to the subject. The virus vector comprising the SGSH nucleic acid is introduced into the cell, and the cell is administered to the subject, where the nucleic acid can be expressed.

### Subjects, Pharmaceutical Formulations, and Modes of Administration

Virus vectors and capsids according to the present invention find use in both veterinary and medical applications. Suitable subjects include both avians and mammals. The term “avian” as used herein includes, but is not limited to, chickens, ducks, geese, quail, turkeys, pheasant, parrots, parakeets, and the like. The term “mammal” as used herein includes, but is not limited to, humans, non-human primates, bovines, ovines, caprines, equines, felines, canines, lagomorphs, etc. Human subjects include neonates, infants, juveniles and adults.

In particular embodiments, the present invention provides a pharmaceutical composition comprising a virus vector of the invention, in particular an AAV particle, in a pharmaceutically acceptable carrier and, optionally, other medicinal agents, pharmaceutical agents, stabilizing agents, buffers, carriers, adjuvants, diluents, etc. For injection, the carrier will typically be a liquid. For other methods of administration, the carrier may be either solid or liquid. For inhalation administration, the carrier will be respirable, and optionally can be in solid or liquid particulate form.

Injectables can be prepared in conventional forms, either as liquid solutions or suspensions, solid forms suitable for solution or suspension in liquid prior to injection, or as emulsions. Alternatively, one may administer the virus vector and/or virus capsids of the invention in a local manner, for example, in a depot or sustained-release formulation. Further, the virus vector and/or virus capsid can be delivered adhered to a surgically implantable matrix (e.g., as described in U.S. Patent Publication No. 2004-0013645).

By “pharmaceutically acceptable” it is meant a material that is not toxic or otherwise undesirable, i.e., the material may be administered to a subject without causing any undesirable biological effects.

One aspect of the present invention is a method of expressing SGSH in a cell in vitro by contacting the cell with the virus vector of the invention so that the virus vector is introduced into the cell and SGSH is expressed by the cell. The virus vector may be introduced into the cells at the appropriate multiplicity of infection according to standard transduction methods suitable for the particular target cells. Titers of virus vector to administer can vary, depending upon the target cell type and number, and the particular virus vector, and can be determined by those of skill in the art without undue experimentation. In representative embodiments, at least about 103 infectious units, more preferably at least about 105 infectious units are introduced to the cell.

In another aspect of the present invention is a method of increasing secretion of SGSH from a cell by contacting the cell with an effective amount of the virus vector of the invention. Secretion of SGSH from the cell may be assessed as described herein or any other suitable method. A cell exhibits an increase in the secretion of SGSH if the cell exhibits a higher amount of secreted SGSH relative to the secretion of SGSH after contacting the cell with an AAV particle comprising a nucleic acid comprising the wild-type sequence for SGSH.

The cell(s) into which the virus vector is introduced can be of any type. Moreover, the cell can be from any species of origin, as indicated above.

The virus vector can be introduced into cells in vitro for the purpose of administering the modified cell to a subject. In particular embodiments, the cells have been removed from a subject, the virus vector is introduced therein, and the cells are then administered back into the subject. Methods of removing cells from subject for manipulation ex vivo, followed by introduction back into the subject are known in the art (see, e.g., U.S. Pat. No. 5,399,346). Alternatively, the recombinant virus vector can be introduced into cells from a donor subject, into cultured cells, or into cells from any other suitable source, and the cells are administered to a subject in need thereof (i.e., a “recipient” subject).

Suitable cells for ex vivo gene delivery are as described above. Dosages of the cells to administer to a subject will vary upon the age, condition and species of the subject, the type of cell, the nucleic acid being expressed by the cell, the mode of administration, and the like. Typically, at least about 10² to about 10⁸ cells or at least about 10³ to about 10⁶ cells will be administered per dose in a pharmaceutically acceptable carrier. In particular embodiments, the cells transduced with the virus vector are administered to the subject in a treatment effective or prevention effective amount in combination with a pharmaceutical carrier.

A further aspect of the invention is a method of delivering or administering the virus vector to subjects. Administration or delivery of the virus vectors according to the present invention to a human subject or an animal in need thereof can be by any means known in the art. Optionally, the virus vector is delivered in a treatment effective or prevention effective dose in a pharmaceutically acceptable carrier.

Dosages of the virus vector to be administered to a subject depend upon the mode of administration, the disease or condition to be treated and/or prevented, the individual subject's condition, the particular virus vector, and the nucleic acid to be delivered, and the like, and can be determined in a routine manner. Exemplary doses for achieving therapeutic effects are titers of at least about 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸ transducing units, optionally about 10⁸ to about 10¹⁵ transducing units.

In particular embodiments, more than one administration (e.g., two, three, four or more administrations) may be employed to achieve the desired level of gene expression over a period of various intervals, e.g., daily, weekly, monthly, yearly, etc.

Exemplary modes of administration include parenteral (e.g., intravenous, subcutaneous, intradermal, intramuscular [including administration to skeletal, diaphragm and/or cardiac muscle], intrathecal, oral, rectal, transmucosal, topical, intranasal, inhalation (e.g., via an aerosol), buccal (e.g., sublingual), vaginal, intraocular, transdermal, in utero (or in ovo), intradermal, intrapleural, intracerebral, and intraarticular), topical (e.g., to both skin and mucosal surfaces, including airway surfaces, and transdermal administration), intro-lymphatic, and the like, as well as direct tissue or organ injection (e.g., to liver, skeletal muscle, cardiac muscle, diaphragm muscle or brain). The most suitable route in any given case will depend on the nature and severity of the condition being treated and on the nature of the particular vector that is being used.

In some embodiments, the viral vector is administered directly to the CNS, e.g., the brain or the spinal cord. Any method known in the art to administer vectors directly to the CNS can be used. The vector may be introduced into the spinal cord, brainstem (medulla oblongata, pons), midbrain (hypothalamus, thalamus, epithalamus, pituitary gland, substantia nigra, pineal gland), cerebellum, telencephalon (corpus striatum, cerebrum including the occipital, temporal, parietal and frontal lobes, cortex, basal ganglia, hippocampus and amygdala), limbic system, neocortex, corpus striatum, cerebrum, and inferior colliculus. The vector may also be administered to different regions of the eye such as the retina, cornea or optic nerve. The vector may be delivered into the cerebrospinal fluid (e.g., by lumbar puncture) for more disperse administration of the vector.

In some embodiments, the viral vector is administered by both intravenous and intrathecal administration. The combination of routes ensures that normal levels of SGSH are achieved in the brain while lowering the total amount of vector that needs to be administered by each route and in total.

Delivery to a target tissue can also be achieved by delivering a depot comprising the virus vector. In representative embodiments, a depot comprising the virus vector is implanted into the tissue or the tissue can be contacted with a film or other matrix comprising the virus vector. Such implantable matrices or substrates are described in U.S. Pat. No. 7,201,898.

In particular embodiments, a virus vector according to the present invention is administered systematically, e.g., intravenously, to treat, delay the onset of and/or prevent symptoms associated with MPS IIIA.

Thus, as one aspect, the invention further encompasses a method of delivering SGSH to a subject, comprising administering to the subject an effective amount of an AAV particle that expresses SGSH, thereby delivering SGSH to the subject.

In another aspect, the invention further encompasses a method of treating, delaying the onset of, and/or preventing MPS IIIA or one or more symptoms associated with MPS IIIA in a subject in need thereof, comprising administering to the subject a therapeutically effective amount of an AAV particle that expresses SGSH, thereby treating, delaying the onset of, and/or preventing MPS IIIA or one or more symptoms associated with MPS IIIA in the subject. In some embodiments, treatment, delay of onset, and/or prevention of MPS IIIA or one or more symptoms associated with MPS IIIA is based upon a comparison to a subject not receiving treatment with the AAV particle.

In the methods of the invention, the subject may be one has been diagnosed with MPS IIIA or is suspected of having MPS IIIA. In certain embodiments, the subject is an infant or child, e.g., less than 18 years old, e.g., less than 18, 17, 16, 15, 14, 13, 12, 11, 10, 9, 8, 7, 6, 5, 4, 3, or 2 years old. In some embodiments, the subject has not developed symptoms of MPS IIIA. Early signs and symptoms of MPS IIIA include frequent ear and throat infections or bowel problems, though most common are mild developmental delay or delayed speech. Behavioral problems often worsen with affected children becoming restless, hyperactive, destructive, anxious, impulsive, fearless, or aggressive. Children with MPS IIIA may have an increased tendency to chew on objects or put things in their mouth (be hyperoral). Sleep disturbances are also very common in children with MPS IIIA. This condition causes progressive intellectual disability and the loss of previously acquired skills (developmental regression or dementia). In later stages of the disorder, people with MPS IIIA may develop seizures, loss of mobility, and movement disorders. Administration of a virus vector encoding SGSH of the invention provides for both enhanced expression and enhanced secretion of SGSH thereby improving upon the treatment or delay of the onset of MPS IIIA.

Having described the present invention, the same will be explained in greater detail in the following examples, which are included herein for illustration purposes only, and which are not intended to be limiting to the invention.

### Example 1

### Development of Improved rAAV-hSGSH Vectors

rAAV Vector Product:

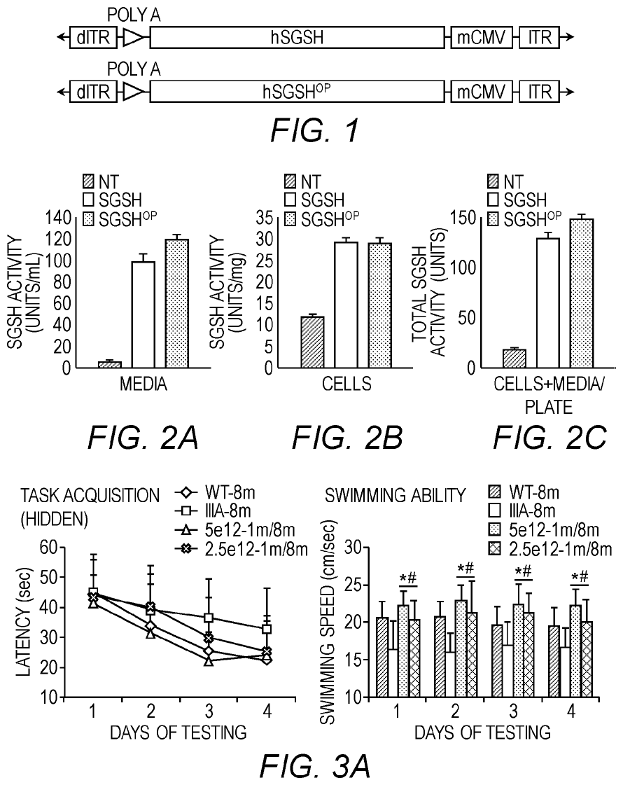

To develop more effective gene therapy products for treating MPS IIIA, two new second-generation scAAV9 vector constructs were developed using a mCMV promoter to drive the expression of hSGSH. The 2^{nd} generation scAAV vector genomes contain only minimal elements required for transgene expression, including a WT AAV2 inverted terminal repeat (ITR), an AAV2 terminal repeat with deletion of terminal resolution site to force generation of self-complementary dimeric genomes (dTR); human SGSH coding sequence cDNA (hSGSH) or codon-optimized hSGSH cDNA (hSGSH^{op}) and SV40 Poly A signal, controlled by a truncated miniature CMV promoter (mCMV). FIG. 1 Illustrates the structures of scAAV-mCMV-hSGSH and scAAV-mCMV-hSGSH^{op} viral vector genomes.

Codon-Modification Enhanced Expression and Secretion of rSGSH:

To assess the impacts of codon-modification on the transgene product, the scAAV-CMV-hSGSH^{op} constructs (ptrs-mCMV-hSGSH^{op}) were tested in vitro in HEK293 cells by transfection, in comparison to the scAAV-mCMV-hSGSH vector construct (ptrs-mCMV-hSGSH). ptrs-mCMV-hSGSH^{op} resulted in significant increases in SGSH activity in the media (FIG. 2A), while there was no difference in SGSH activity levels in cell lysates (FIG. 2B), compared to ptrs-mCMV-hSGSH. These data demonstrate that the codon-optimization enhanced the secretion and overall expression of rSGSH. It is therefore believed that the scAAV-hSGSH^{op} vector may have added therapeutic benefits for treating MPS IIIA over the scAAV-hSGSH vector product, by improved bystander effects of rSGSH due to the enhanced rSGSH secretion and expression.

Notably, in addition to the optimized hSGSH sequence of SEQ ID NO:1 shown to increase expression and secretion, 8 other optimized hSGSH sequences (SEQ ID NOS: 8-15) were prepared and tested for expression. These 8 other sequences either did not enhance SGSH expression or actually exhibited reduced expression to <80% of the wild-type vector.

Functional Therapeutic Benefits of scAAV9-mCMV-hSGSH Vector for Treating MPS IIIA in Mice Via Systemic Delivery:

To determine the therapeutic potential of the scAAV9-mCMV-hSGSH gene therapy vector, the vector was tested in MPS IIIA mice at age 1 m, 3 m, or 6 m, with an IV injection of the vector at 2.5×10¹² vg/kg (n=18), 5×10¹² vg/kg (n=22), or 1×10¹³ vg/kg (Cohort 1-4). Behavior tests were conducted in all mice in a hidden task in a Morris water maze at 8 m and/or 12 m of age (n=6-18/group). Tissue analyses were performed for SGSH enzyme activity, GAG contents, histopathology, and biodistribution at 1 m pi, 8 m of age (n=4-5/group) or humane endpoint. Subsets of mice from each group were observed for longevity (n=6-13). To assess the potential acute toxicity, small groups of animals (Cohort 5-7) were treated with the vector at higher doses, 3×10¹³ vg/kg or 5×10¹³ vg/kg. Controls were combined randomly assigned non-treated MPS IIIA and wild-type littermates. Table 2 summarizes the overall experimental design.

TABLE 2
Study design: systemic rAAV9-CBA-hSGSH delivery in MPS IIIA mice

| | | | No. mice (n) | | | | | | | |
|---|---|---|---|---|---|---|---|---|---|---|
| | Vector | Injection | | Behavior | | Necropsy | | | | |
| | dose | Age | | Age | Age | 5 d | 1 m | Age | | |
| Cohorts | (vg/kg) | (m) | Total | 8 m | 12 m | pi | pi | 8 m | End | Longevity |
| 1. MPS | 2.5 × 10¹² | 1 | 18 | 12 | — | — | 5 | 5 | 5 | 8 |
| 2. MPS | 5 × 10¹² | 1 | 22 | 18 | — | — | 4 | 5 | 9 | 13 |
| 3. MPS | 1 × 10¹³ | 3 | 9 | 5 | 5 | — | 4 | — | 5 | 5 |
| 4. MPS | 1 × 10¹³ | 6 | 6 | 6 | 5 | — | — | — | 6 | 6 |
| 5. MPS* | 5 × 10¹³ | 3-4 | 4 | — | — | — | 4 | — | | 0 |
| 6. WT* | 5 × 10¹³ | 4 | 8 | — | — | 4 | 4 | — | 4 | 0 |
| 7. WT* | 3 × 10¹³ | 2 | 3 | — | — | 3 | — | — | 3 | 0 |
| 8. MPS** | — | — | 63 | 36 | — | — | 5 | 5 | | 53 |
| 9. WT** | — | — | 75 | 75 | 6 | — | 5 | 5 | | 53 |

*Non-GLP toxicology testing;
**Controls combined from multiple experiments;
—: not performed.

### Significantly Behavioral Improvement and Extension in Survival:

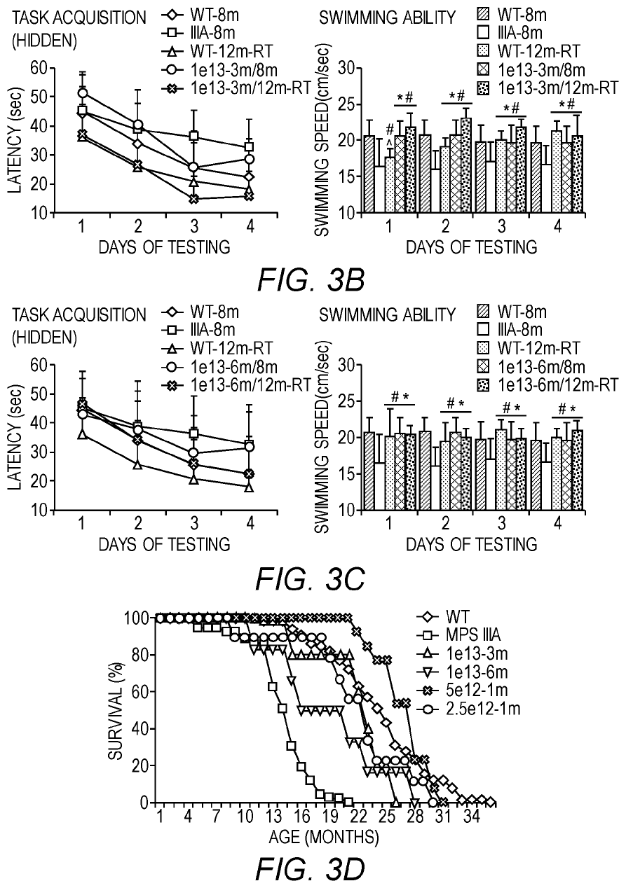

To assess the functional neurological benefits of scAAV9-mCMV-hSGSH via an IV delivery, the vector-treated mice were tested for performance in a hidden task in a Morris water maze at 8 m (FIGS. 3A-3C) and/or 12 m of age (FIGS. 3B-3C). The vector treatments led to significant improvement to WT levels in latency to find a hidden platform and swimming ability, in MPS IIIA mice treated at age 1 m with 2.5×10¹² vg/kg (n=12) or 5×10¹² vg/kg (n=18) when tested at age 8 m, indicating the correction of cognitive and motor function. These data further support the notion of functional dose threshold of an IV delivery of scAAV9-mCMV-hSGSH, because of the bystander effect of SGSH. MPS IIIA mice treated at age 3 m (n=8) or 6 m (n=6) with 1×10¹³ vg/kg vector showed normalized swimming ability, but only partial correction in latency to find the hidden platform, when tested at age 8 m (FIGS. 3B-3C). However, when re-tested at age 12 m, animals treated at age 3 m (n=5) or 6 m (n=5) showed latency and swimming ability similar to WT mice (n=6) (FIGS. 3B-3C). Notably, both WT and MPS IIIA mice treated with the vector at age 3 m or 6 m showed further improvement in latency to find the hidden platform, rather than decline in ability, when retested at age 12 m, compared to that of the first test at age 8 m (FIGS. 3B-3C). These data indicate that the vector treatment led to the correction of cognitive and motor function in MPS IIIA animals.

### Rapid and Persistent Expression of Functional SGSH Throughout the CNS and Periphery:

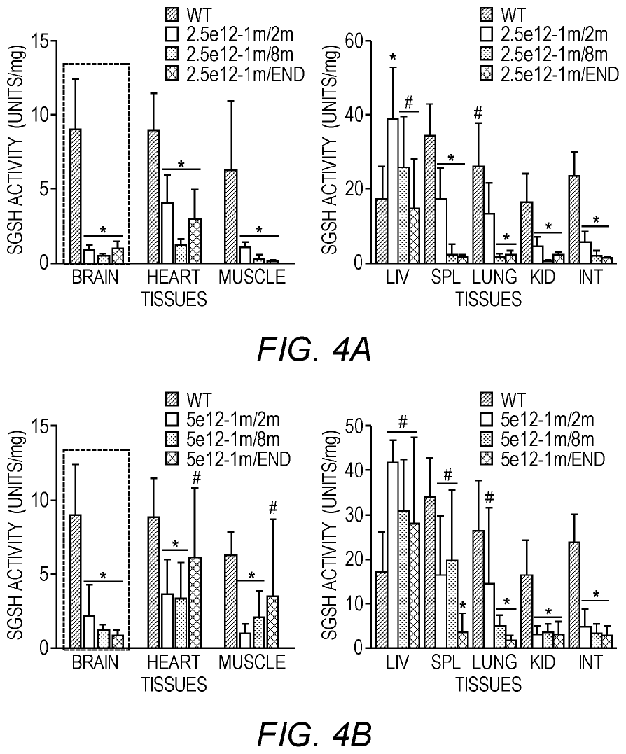

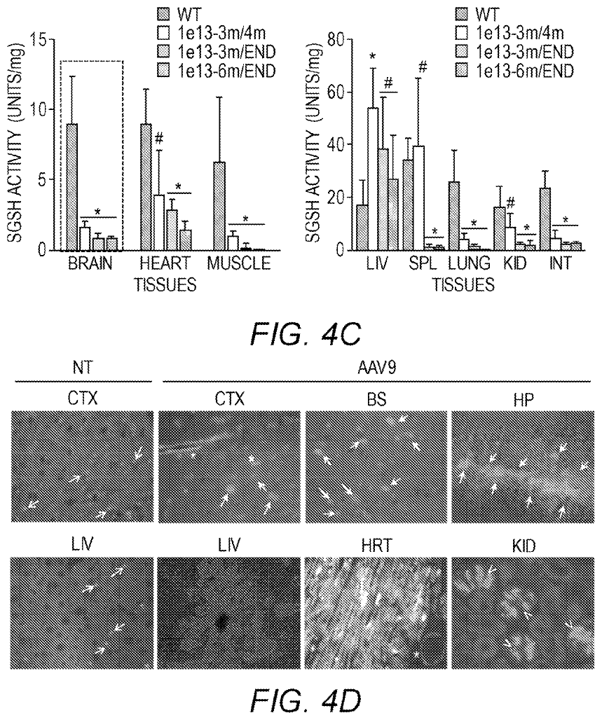

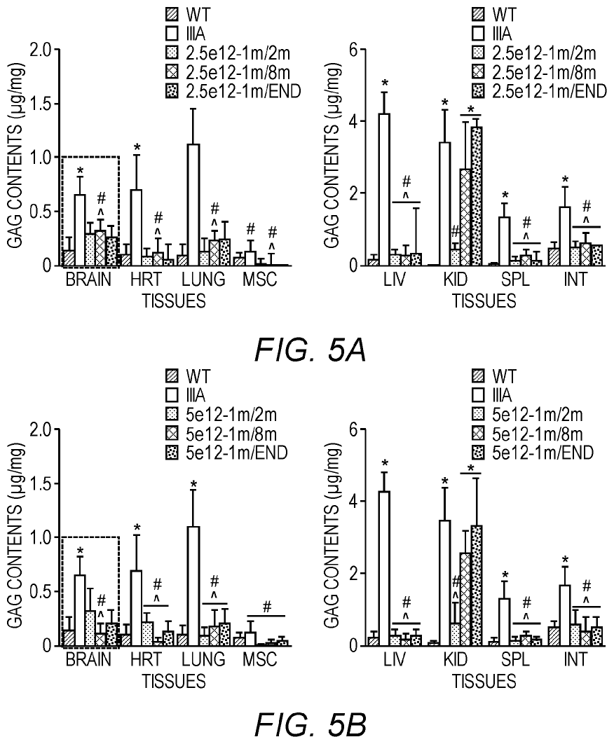

To evaluate the persistence and levels of scAAV9-mediated rSGSH expression, tissues were assayed for SGSH activity at 1 m pi, age 8 m, and the humane endpoint. The results showed SGSH activity at above normal levels in the liver, at close to normal levels in heart, spleen, and lung, and at subnormal levels in the brain, intestine, skeletal muscle, and kidney, at 1 m pi in MPS IIIA mice treated with 2.5×10¹² vg/kg, 5×10¹² vg/kg, or 1×10¹³ vg/kg vector (FIGS. 4A-4C). Importantly, among all vector-treated MPS IIIA mice, brain SGSH activity was detected at 5.5-18.2% of WT levels. Further, SGSH activity levels persisted in the brain and the majority of tested somatic tissues, while a significant decrease in SGSH activity was observed in the liver, spleen, and lung over time from 1 m pi to the endpoint (FIGS. 4A-4C). Dose response was observed only in the liver and possibly in the spleen (FIGS. 4A-4B). These results demonstrate that the scAAV9-mediated rSGSH expression is quick and persistent, and the rSGSH is enzymatically functional. The decrease in SGSH activity in liver, spleen and lung over time may be due to the slow cell turnover and the predominantly episomal status of AAV vector.

Further, tissues were also assayed for hSGSH by immunofluorescence (IF) staining, to determine the distribution of the recombinant enzyme. The rSGSH expression was observed in cells throughout the CNS and peripheral tissues in vector-treated MPS IIIA mice (FIG. 4D), correlating with the SGSH activity levels (FIGS. 4A-4C).

### Lifelong Clearance of Lysosomal Storage Pathology and Correction of Astrocytosis:

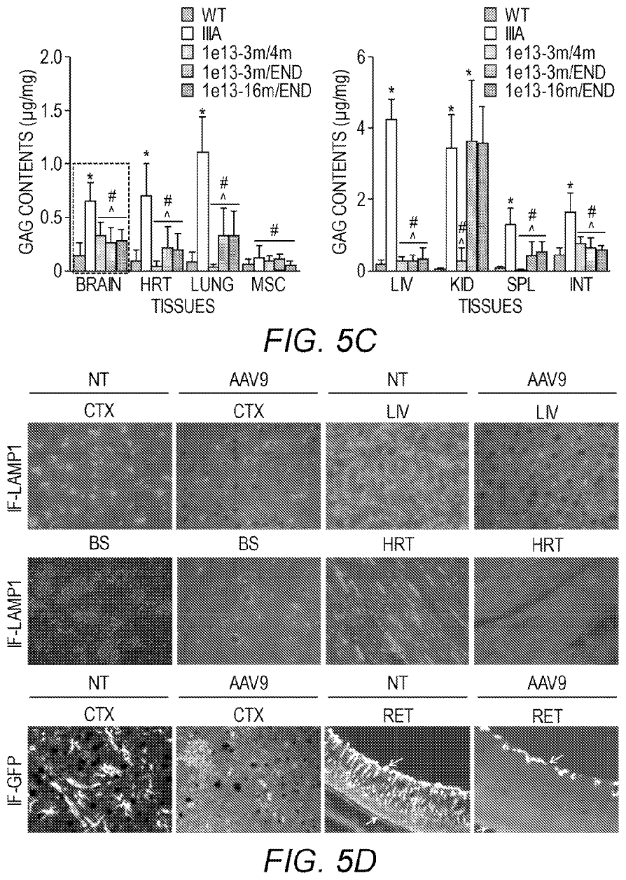

To further assess the functionality of scAAV9-mediated rSGSH, brain and multiple peripheral tissues were assayed for GAG contents at 1 m pi, 8 m of age or the humane endpoint. As shown in FIGS. 5A-5C, the vector treatments at all 3 tested doses led to significant reduction of GAG contents to normal levels in the majority of tested tissues, including the brain and 7 somatic tissues, with the exception of kidney. Importantly, the normalized tissue GAG content was observed at all testing time points, from 1 m pi to the humane endpoint, in all MPS IIIA mice treated with the vector. These data further support that the scAAV9-mediated rSGSH is functional, leading to rapid clearance of GAG contents, and the correction and reversal of GAG storage in the CNS and periphery. Importantly, the complete clearance of tissue GAG storage persisted to the endpoint (FIGS. 5A-5C), correlating with the long-term tissue rSGSH expression (FIGS. 4A-4C). The clearance of lysosomal storage pathology was further confirmed by IF staining for lysosomal associated membrane protein 1 (LAMP1), showing the reduction of LAMP1 in the brain and peripheral tissues (FIG. 5D).

In addition, IF staining showed the clearance of GFAP-positive signals in the brain and retina (FIG. 5D), indicating the correction of astrocytosis, a hallmark of neuroinflammation in the CNS and optical nervous system (ONS).

Differential Biodistribution of rAAV9-mCMV-hSGSH Vector Genome in the CNS and Periphery:

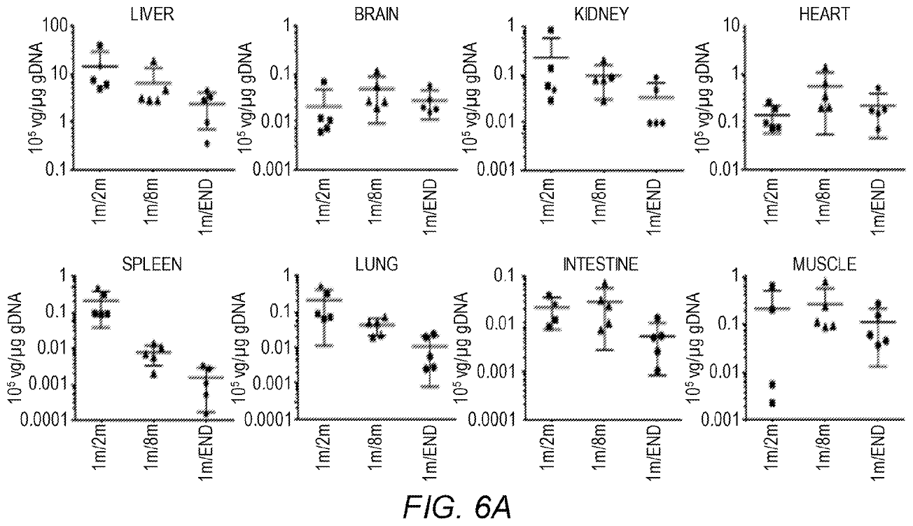

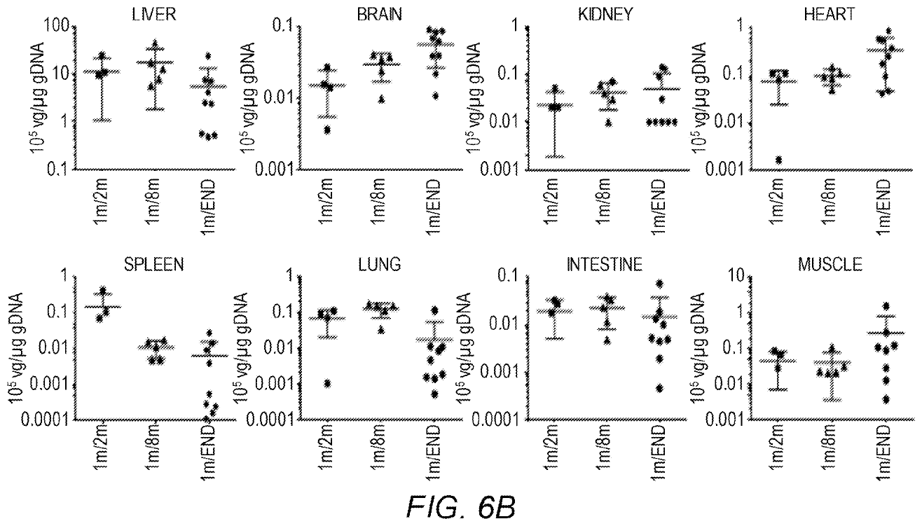

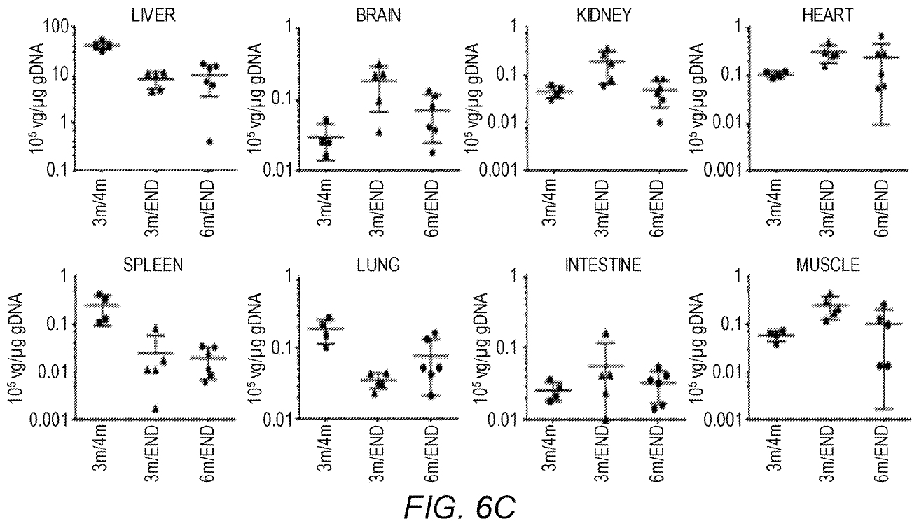

To determine the biodistribution of scAAV9-mCMV-hSGSH following an IV infusion, total tissue DNA samples was assayed using qPCR to quantitate scAAV9-hSGSH vector genome in tissues at different time points after vector injection (n≥4/group). We observed differential biodistribution of the vector DNA in tissues in MPS IIIA mice, with the highest vg levels detected in the liver, followed by heart, skeletal muscle, lung, intestine, kidney, spleen, and brain (FIGS. 6A-6D). Differences in tissue vg copy numbers were observed among MPS IIIA mice receiving scAAV9-mCMV-hSGSH vector treatments. The vector genome copies persisted in the majority of tissues, though decreases of vector genome were observed in liver, spleen, and lung over time.

Similar differential biodistribution profiles were also observed in the small non-GLP toxicology testing in MPS IIIA (Cohort 5) and WT (Cohort 6, 7) mice that received an IV injection of scAAV9-mCMV-hSGSH at high doses (Table 2). The results showed dose response of tissue vg in the majority of tissues in WT mice at 1 m pi.

### No Observable Adverse Events and Detectable Toxicity:

All mice used in this study were observed for potential adverse events following an IV injection of scAAV9-mCMV-hSGSH. No adverse events were seen in MPS IIIA mice treated with the vector at 2.5×10¹² vg/kg-1×10¹³ vg/kg in the efficacy studies during the entire experimental period until humane endpoints. Further, no acute side effects were observed in MPS IIIA and WT mice treated at a higher vector dose (3×10¹³ vg/kg or 5×10¹³ vg/kg) in short non-GLP-toxicology studies. In addition, brain and 7 somatic tissues from all mice in this study were processed for histopathology examination by a certified veterinary pathologist, and the results showed no detectable abnormality associated with the vector treatments in any animal subject. These data indicate that scAAV9-mCMV-hSGSH via an IV delivery was safe and did not induce systemic toxicity at tested doses.

### Example 2

### Preclinical Assessment of scAAV9-mCMV-hSGSH^{op} Gene Delivery for Treating MPS IIIA

To assess the therapeutic potential, the scAAV9-mCMV-hSGSH^{op} vector was tested in 1-2 m-old MPS IIIA mice (n=12-15/group) by an IV injection at 5×10¹² vg/kg, 1×10¹³ vg/kg, or 2×10¹³ vg/kg, or by combined IV (2×10¹² vg/kg) and intrathecal (IT, 1×10¹² vg/kg) delivery into the cisterna magna. Non-treated MPS IIIA and WT mice were used as controls. Necropsy was performed at 1 m post injection (pi) for tissue analyses (n=4/group). The animals are currently under long-term observation and will be tested for behavior performance in a hidden task in a Morris water maze (n≥8/group). A subset of mice from each cohort will be observed for longevity. Necropsy will also be performed at 8 m pi or humane endpoint for tissue analyses to assess rSGSH expression, the correction of lysosomal storage pathology and histopathology.

### Rapid and Persistent Expression of Functional SGSH Throughout the CNS and Periphery:

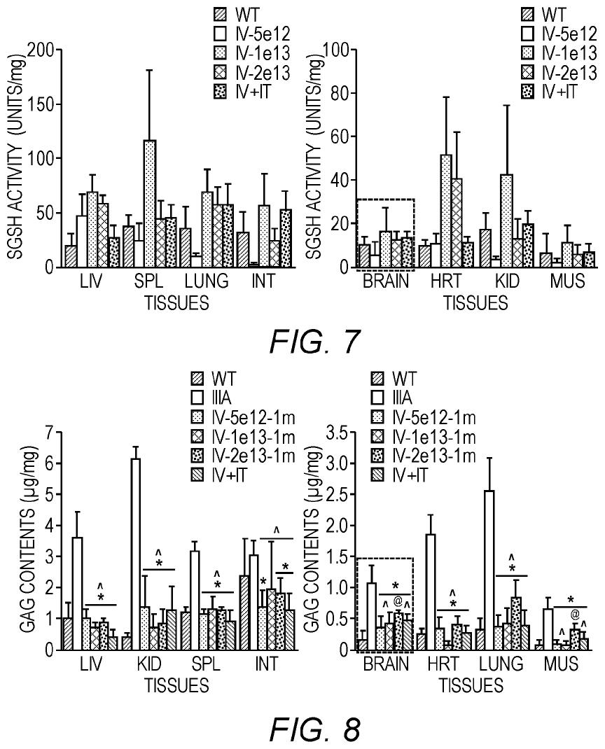

To evaluate the scAAV9-hSGSH^{op} mediated rSGSH expression, tissues were assayed for SGSH activity at 1 m pi. The results showed SGSH activity at above normal levels in the liver and heart and at or close to normal levels in the brain, spleen, lung, intestine, kidney, and skeletal muscle in MPS IIIA mice treated with an IV injection of 1×10¹³ vg/kg and 2×10¹³ vg/kg vector (FIG. 7). In mice treated with an IV injection of 5×10¹² vg/kg vector, SGSH activity was detected at above WT levels in the liver, at WT level in heart, and subnormal levels in brain, spleen, lung, intestine, kidney, and skeletal muscle, with brain SGSH activity at 8-50% of WT levels (FIG. 7). Dose response was observed in the brain and liver, and possibly in the lung and heart (FIG. 7) in mice receiving a systemic scAAV9-hSGSH^{op} delivery. Notably, scAAV9-hSGSH^{op} via an IV injection appeared to mediate higher tissue SGSH activity levels in MPS IIIA mice (FIG. 7), compared to scAAV9-hSGSH (FIGS. 4A-4C). These data further demonstrate in vivo the enhanced rSGSH expression/secretion by codon-optimization.

Further, SGSH activity was detected at normal levels in the brain and all of the tested peripheral tissues (FIG. 7), in animals treated with the combined IV (2×10¹² vg/kg) and IT (1×10¹² vg/kg) vector delivery.

These results demonstrate that the scAAV9-hSGSH^{op}-mediated rSGSH expression is rapid, and the rSGSH is enzymatically functional, supporting the therapeutic potential for treating MPS IIIA in humans.

### Rapid Clearance of Lysosomal GAG Storage:

To assess the functionality of scAAV9-hSGSH^{op}-mediated rSGSH, brain and multiple peripheral tissues were assayed for GAG contents at 1 m pi. As shown in FIG. 8, a significant reduction of GAG contents to normal levels was seen in virtually all tested tissues from MPS IIIA mice that were treated with an IV vector injection at 5×10¹² vg/kg, 1×10¹³ vg/kg, or 2×10¹³ vg/kg, or with a combined IV (2×10¹² vg/kg) and IT (1×10¹² vg/kg) vector infusion. These results further demonstrate that the scAAV9-hSGSH^{op}-mediated rSGSH is functional, leading to rapid clearance of GAG storage in the CNS and periphery, supporting the therapeutic potential of the scAAV9-mCMV-hSGSH^{op} vector via an IV or a combined IV and IT delivery.

Differential Biodistribution of rAAV9-mCMV-hSGSH^{op} Vector Genome in the CNS and Periphery:

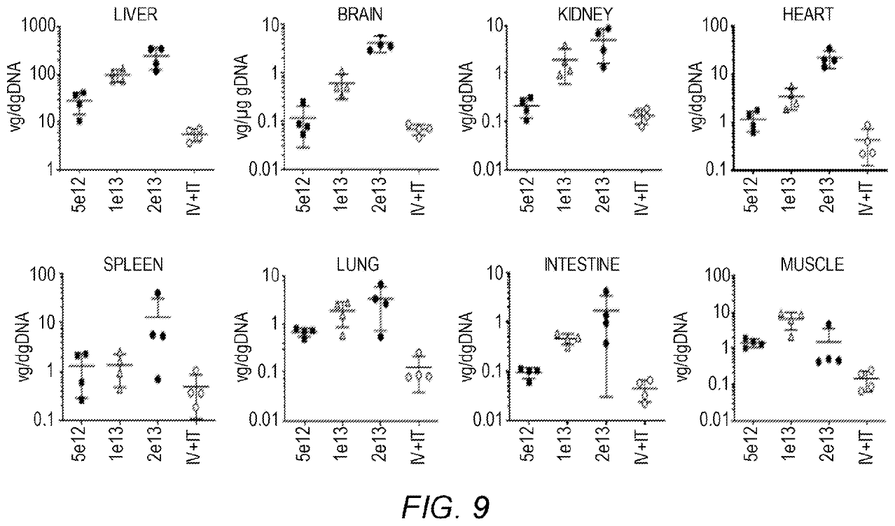

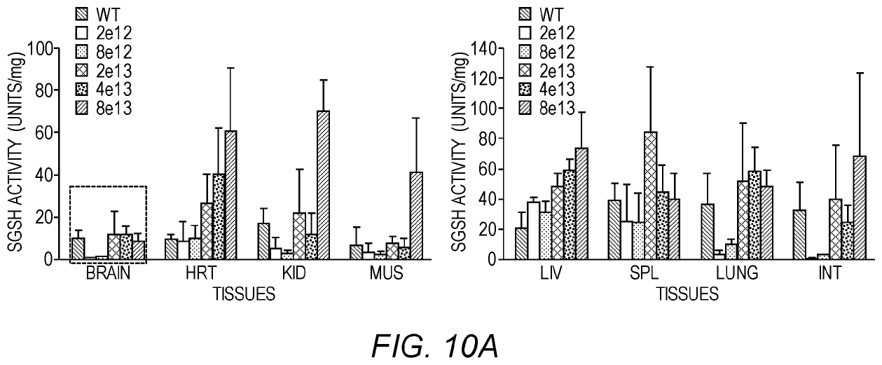

To determine the biodistribution of scAAV9-mCMV-hSGSH^{op} following an IV or a combined IV and IT infusion, total tissue DNA samples were assayed using qPCR to quantitate scAAV9-hSGSH^{op} vector genome in tissues (n=4/group). The results showed differential biodistribution of the vector DNA in tissues in the vector treated MPS IIIA mice, with the highest vg levels detected in the liver, followed by heart, skeletal muscle, lung, intestine, kidney, spleen, and brain (FIG. 9). A dose response of vg biodistribution was observed in the majority of the tested tissues in mice treated with an IV vector injection. Not surprisingly, relatively lower vg copy levels were detected in MPS IIIA mice that received a combined IV (2×10¹² vg/kg) and IT (1×10¹² vg/kg) vector infusion (FIG. 9).

In summary, two safe and potentially more effective new second-generation scAAV9 gene replacement therapy products were developed for the treatment of MPS IIIA. The pre-clinical study demonstrates that a systemic delivery of the scAAV9-mCMV-hSGSH vector targeting the root cause can prevent and reverse the broad neurological and somatic manifestations of the disease. It was demonstrated again here that the efficacy and safety profiles of systemic rAAV9 gene delivery are highly reproducible. These in vitro studies showed that codon-optimization of hSGSH improved not only the expression but also the secretion of rSGSH, suggesting the potential of enhanced bystander effects. Pre-clinical studies of scAAV9-mCMV-hSGSH^{op} further indicate the enhanced rSGSH expression in MPS IIIA mice, leading to rapid correction of lysosomal GAG storage in the CNS and peripheral organs following a systemic or a combined IV and IT delivery. These data further support the potential of the demonstrated AAV9 vector platform for the effective translation of rAAV9 gene therapy products to clinical application to benefit MPS IIIA, as well as broad neurogenetic disease patient populations.

### Example 3

### Preclinical Dose Assessment of scAAV9-mCMV-hSGSH^{op} Gene Delivery for Treating MPS IIIA

To further assess different doses of the disclosed scAAV9-mCMV-hSGSH^{op} gene delivery, the vector was tested in the MPS IIIA mouse model. Table 3 summarizes the experimental designs. MPS IIIA mice were treated at age 1 m with an IV injection of scAAV9-mCMV-hSGSH^{op} vector at 2e12 vg/kg, 8e12 vg/kg, 2e13 vg/kg, 4e13 vg/kg, or 8e13 vg/kg. Tissue analyses were or will be performed at 1 m pi and/or 7 m pi (n=3-5/group), or humane endpoints. The animals were or will be tested for behavior performance in a hidden task in a Morris water maze at 8 m of age. Subsets of these groups remain under observation for longevity. Randomly assigned non-treated MPS IIIA mice and WT littermates were used as controls.

TABLE 3
Study design: systemic scAAV9-mCMV-hSGSH
^{op} gene delivery in MPS IIIA mice

| | | N | | | | | |
|---|---|---|---|---|---|---|---|
| | | | | Tissue analyses | | | |
| | Vector | | Behavior | 1 m | 7 m | | Lon- |
| Cohorts | (vg/kg) | Total | Testing** | pi | pi | End | gevity |
| MPS IIIA | 2e12 | 14 | 10\hat{ } | 4 | — | +\hat{ } | 8 |
| MPS IIIA | 8e12 | 15 | 11 | 4 | 4\hat{ } | +\hat{ } | 7 |
| MPS IIIA | 2e13 | 12 | 8 | 4 | 4\hat{ } | +\hat{ } | 4 |
| MPS IIIA | 4e13 | 12 | 8 | 4 | — | +\hat{ } | 8 |
| MPS IIIA | 8e13 | 9 | — | 4 | — | +\hat{ } | 5 |
| MPS IIIA* | — | 53 | 36 | 4 | 4 | — | 45 |
| WT* | — | 75 | 38 | 4 | 4 | — | 65 |

*Accumulated over time;
**Behavior testing at age 8 m;
\hat{ }to be performed.

Quick and Persist Restoration of SGSH Enzyme Activity in the CNS and Peripheral Tissues after an IV scAAV9-mCMV-hSGSH^{op} Delivery:

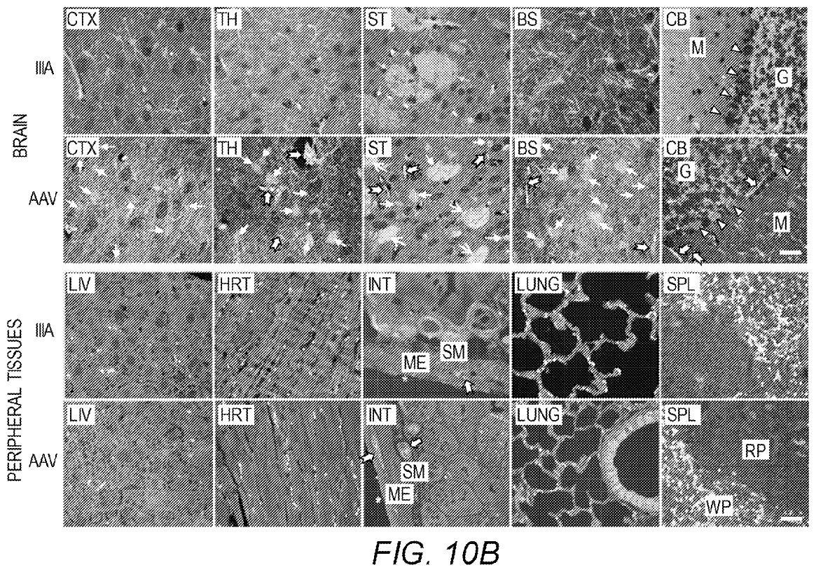

To assess the levels and function of AAV-mediated rSGSH expression, brain and multiple tissues from vector-treated mice of all dose cohorts were assayed for SGSH activity at 1 m pi. As results, SGSH activity was detected above normal levels in the liver, and at normal or close to normal levels in spleen and muscle in all dose groups (FIG. 10A). Heart SGSH activity levels were observed at WT levels in mice treated with 2e12 vg/kg and 8e12 vg/kg vector, and at above normal levels in mice receiving higher doses (FIG. 10A). Normal or above normal levels of SGSH activity were detected in kidney, lung, and intestine in mice treated with 2e13 vg/kg-8e13 vg/kg vector, and at sub normal levels in mice receiving lower doses (FIG. 10A). Notably, the vector treatment resulted in brain SGSH expression at WT levels in the 2e13 vg/kg-8e13 vg/kg dose groups, and sub normal levels of brain SGSH activity in the lower dose groups (FIG. 10A). These results indicate the quick and efficient rSGSH expression in the CNS and somatic tissues. Dose response of AAV-mediated SGSH activity was observed in the majority of tissues (FIG. 10A).

Further, tissues were assayed by immunofluorescence (IF) for hSGSH to visualize the expression and distribution of rSGSH. The results showed rSGSH expression in cells throughout the CNS, cells in myenteric plexus and submucosal plexus, and peripheral tissues in vector-treated MPS IIIA mice at 1 m pi (data not shown) and 7 m pi (FIG. 10B), correlating to tissue SGSH activity levels. FIG. 10B presents IF images of tissues from a mouse of the 2e13 vg/kg group at 7 m pi. These data further demonstrate that the vector treatments led to the quick and persistent restoration of functional SGSH throughout the CNS, in peripheral nervous system (PNS), and broad peripheral organs.

### Quick and Persistent Clearance of Lysosomal GAG Storage in the CNS and Peripheral Tissues:

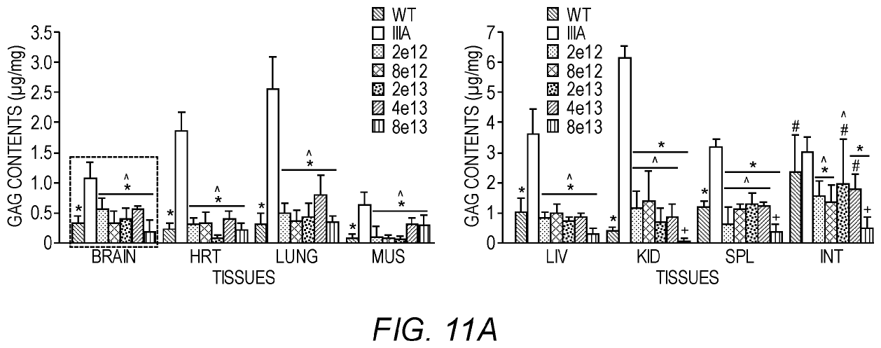

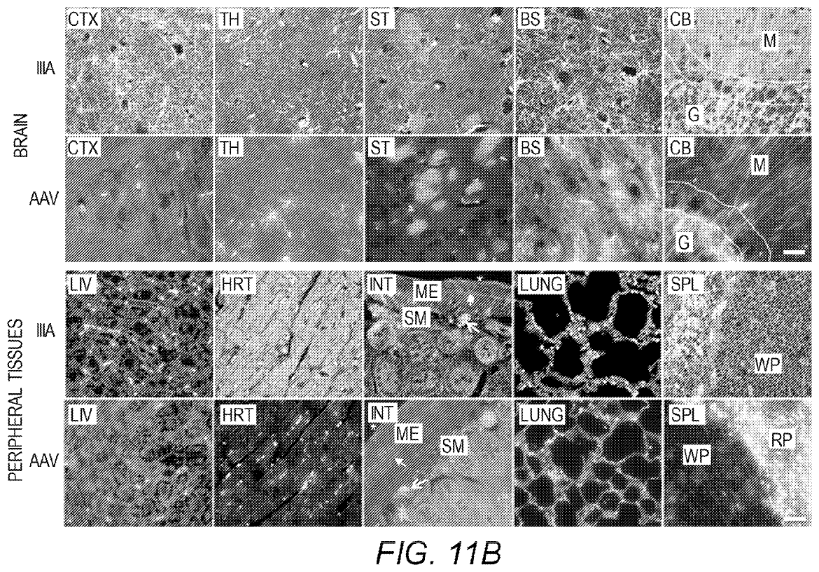

To further assess the functionality of AAV9-mediated rSGSH, brain and multiple peripheral tissues were assayed for GAG contents. As shown in FIG. 11A, the vector treatments at all tested doses led to significant reduction of GAG contents to WT levels in all tested somatic tissues.

IF staining for lysosomal associated membrane protein 1 (LAMP 1) and GFAP were performed on paraffin tissue sections from different dose groups at 1 m pi and 7 m pi, to visualize the impacts of the vector treatment on the lysosomal storage pathology and astrocytosis. The results showed the clearance of LAMP1 and GFAP signals throughout the brain, and in cells of both myenteric plexus and submucosal plexus in the intestine in vector-treated MPS IIIA mice at both 1 m pi (data not shown) and 7 m pi (FIG. 11B). The clearance of LAMP1 were also observed in virtually all tested peripheral tissues at both 1 m pi (data not shown) and 7 m pi (FIG. 11B). FIG. 11B presents IF images of tissues from a mouse of the 2e13 vg/kg group at 7 m pi. These data further support that the scAAV9-hSGSH^{op}-mediated rSGSH is functional, leading to rapid and persistent clearance of lysosomal storage pathology in the CNS and periphery. Further, the vector treatments also resulted in quick and persistent diminishment of astrocytosis in the CNS and PNS (FIG. 10B, FIG. 11B), indicating the correction of neuroinflammation.

Functional Correction of Behavior Deficits and Significant Extension of Survival in MPS IIIA Mice after an IV scAAV9-mCMV-hSGSH^{op} Gene Delivery:

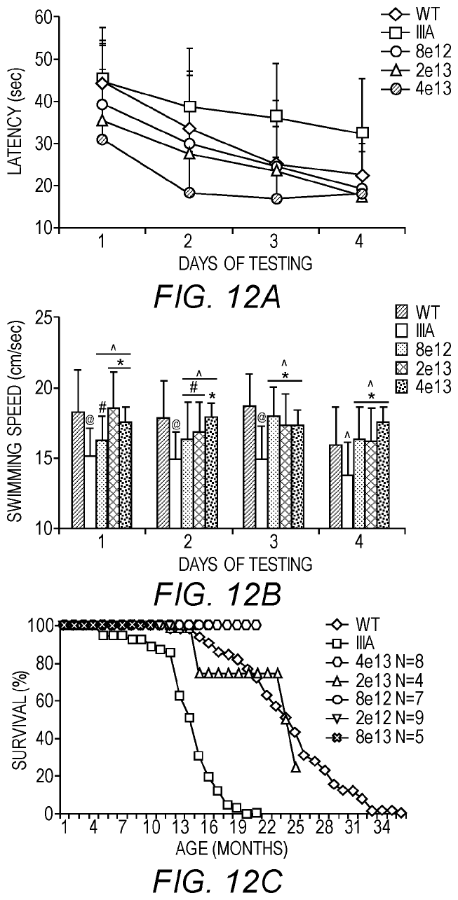

To assess the functional benefits of systemic scAAV9-mCMV-hSGSH gene delivery, 3 groups of AAV9-treated animals were tested for behavior performance in a hidden task in a Morris water maze at age 8 m (Table 3, FIG. 12A-12B). The results showed significant improvement in latency to find a hidden platform (FIG. 12A) and swimming ability (FIG. 12B) in MPS IIIA mice treated with 8e12 vg/kg, 2e13 vg/kg, and 4e13 vg/kg, indicating the correction of cognitive and motor function.

Longevity studies showed a significant extension in survival and the majority of MPS IIIA treated with 8e12 vg/kg, 2e13 vg/kg, and 4e13 vg/kg vector lived/living within the normal range of lifespan (FIG. 12C). While the longevity studies are ongoing, MPS IIIA mice receiving 2e12 vg/kg or 8e13 vg/kg vector appear to live longer than non-treated MPS IIIA mice (FIG. 12C). These data further support the functional neurological benefits of the scAAV9-mCMV-hSGSH vector treatments, as premature death in MPS IIIA is predominantly attributed to the profound neuropathy.

Together, these data demonstrate that a single IV scAAV9-mCMV-hSGSH^{op} gene delivery is functionally beneficial for treating the neurological and somatic disorder MPS IIIA. Based on these data, it is believed that the minimal effective clinical dose is ≤2e12 vg/kg.

Differential Bio-Distribution of Systemically Delivered scAAV9-mCMV-hSGSH^{op} in MPS IIIA Mice:

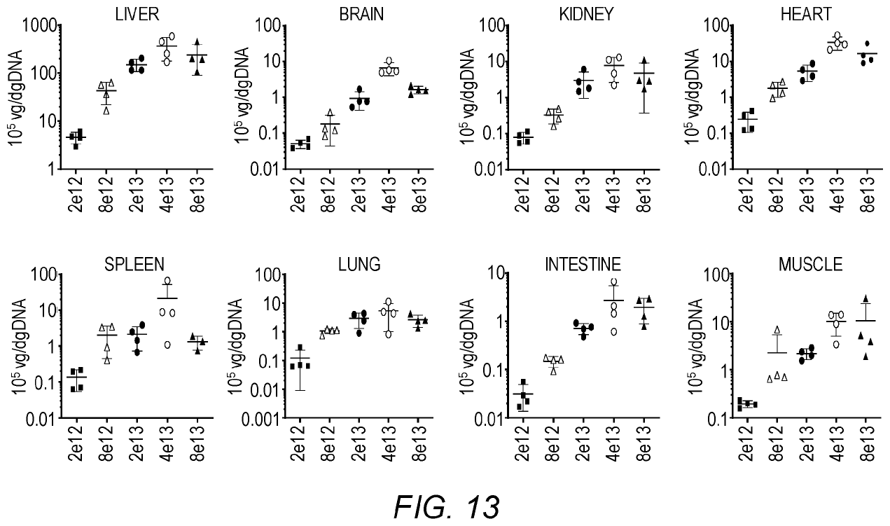

Tissues collected at 1 m pi were assayed for scAAV9-mCMV-hSGSH^{op} vector genome to assess the bio-distribution of the IV-delivered vector in the CNS and periphery. The results showed differential and persistent bio-distribution of the vector in tissues (FIG. 13), correlating to tissue rSGSH activity (FIG. 10A). Dose response of biodistribution was observed in the majority of the tissues.

### No Observable Adverse Events:

To date, no adverse events have been observed in MPS IIIA mice that received the vector treatment, supporting the safe profile of the product.

In summary, the effective gene replacement therapy product scAAV9-mCMV-hSGSH^{op} has been developed and tested at different doses in the MPS IIIA mouse model at age 1 m via a systemic delivery. These data demonstrate that a single IV injection of scAAV9-mCMV-hSGSH vector is safe and functionally beneficial, leading to quick and persistent restoration of SGSH activity, the clearance of lysosomal storage pathology, and correction of the disease in MPS IIIA mice.

The foregoing is illustrative of the present invention and is not to be construed as limiting thereof. The invention is defined by the following claims, with equivalents of the claims to be included therein.

1. A recombinant nucleic acid comprising a sequence encoding human N-sulfoglucosamine sulfohydrolase (SGSH) that is codon-optimized for expression in human cells, wherein the recombinant nucleic acid comprises a nucleotide sequence at least 90% identical to SEQ ID NO:1.

2. The recombinant nucleic acid of claim 1, comprising the nucleotide sequence of SEQ ID NO: 1.

3. A vector comprising the recombinant nucleic acid of claim 1 or 2.

4. An adeno-associated virus (AAV) vector genome comprising the recombinant nucleic acid of claim 1.

5. The AAV vector genome of claim 4, wherein the recombinant nucleic acid is operably linked to a constitutive promoter.

6. The AAV vector genome of claim 5, wherein the constitutive promoter is a miniature CMV promoter.

7. The AAV vector genome of claim 4, wherein the recombinant nucleic acid is operably linked to a SV40 poly (A) signal.

8. The AAV vector genome of claim 4, wherein the AAV vector genome is a self-complementary AAV vector genome.

9. The AAV vector genome of claim 8, comprising a wild-type AAV2 inverted terminal repeat and an AAV2 inverted terminal repeat with deletion of the terminal resolution site.

10. The AAV vector genome of claim 9, comprising a nucleotide sequence at least 90% identical to SEQ ID NO:5.

11. The AAV vector genome of claim 10, comprising the nucleotide sequence of SEQ ID NO:5.

12. A cell in vitro comprising the AAV vector genome of claim 4.

13. An AAV particle comprising the AAV vector genome of claim 4.

14. A method of producing a recombinant AAV particle comprising an AAV capsid, the method comprising:
providing a cell in vitro with nucleic acids comprising AAV Cap and AAV Rep coding sequences, the AAV vector genome of claim 4, and helper functions for generating a productive AAV infection; and
allowing assembly of the recombinant AAV particle comprising the AAV capsid and encapsidating the AAV vector genome.

15. (canceled)

16. The AAV particle of claim 13, wherein the AAV particle is an AAV9 particle.

17. A pharmaceutical formulation comprising the AAV particle of claim 13 and a pharmaceutically acceptable carrier.

18. A method of expressing SGSH in a cell, comprising contacting the cell with an effective amount of the AAV particle of claim 13, thereby expressing SGSH in the cell.

19. A method of increasing secretion of SGSH from a cell, comprising contacting the cell with an effective amount of the AAV particle of claim 13, thereby increasing secretion of SGSH from the cell relative to the secretion of SGSH after contacting the cell with an AAV particle comprising a nucleic acid comprising the wild-type sequence for SGSH.

20. A method of delivering SGSH to a subject, comprising administering to the subject an effective amount of the AAV particle of claim 13, thereby delivering SGSH to the subject.

21. A method of treating or delaying the onset of mucopolysaccharidosis IIIA (MPS IIIA) in a subject in need thereof, comprising administering to the subject a therapeutically effective amount of the AAV particle of claim 13, thereby treating or delaying the onset of MPS IIIA in the subject.

22-25. (canceled)

26. The method of claim 21, wherein the subject is a human subject.

27. The method of claim 21, wherein the subject has been diagnosed with MPS IIIA.


SEQUENCE LISTING (16 sequences)

SEQ ID NO: 1 - DNA, 1509 bp, synthetic construct
  source 1..1509: mol_type = other DNA
atgtcatgccctgtccccgcatgttgtgctttgctccttgtacttgggctttgccgagcgcgtccccggaacgcactgctgctcctcgcggatgacggaggctttgagagtggcgcgtacaacaacagcgccatcgccaccccgcacctggacgccttggcccgccgcagcctcctctttcgcaatgccttcacctcggtcagcagctgctctcccagccgcgccagcctcctcactggcctgccccagcatcagaatgggatgtacgggctgcaccaggacgtgcaccacttcaactccttcgacaaggtgcggagcctgccgctgctgctcagccaagctggtgtgcgcacaggcatcatcgggaagaagcacgtggggccggagaccgtgtacccgtttgactttgcgtacacggaggagaatggctccgtcctccaggtggggcggaacatcactagaattaagctgctcgtccggaaattcctgcagactcaggatgaccggcctttcttcctctacgtcgccttccacgacccccaccgctgtgggcactcccagccccagtacggaaccttctgtgagaagtttggcaacggagagagcggcatgggtcgtatcccagactggaccccccaggcctacgacccactggacgtgctggtgccttacttcgtccccaacaccccggcagcccgagccgacctggccgctcagtacaccaccgtcggccgcatggaccaaggagttggactggtgctccaggagctgcgtgacgccggtgtcctgaacgacacactggtgatcttcacgtccgacaacgggatccccttccccagcggcaggaccaacctgtactggccgggcactgctgaacccttactggtgtcatccccggagcacccaaaacgctggggccaagtcagcgaggcctacgtgagcctcctagacctcacgcccaccatcttggattggttctcgatcccgtaccccagctacgccatctttggctcgaagaccatccacctcactggccggtccctcctgccggcgctggaggccgagcccctctgggccaccgtctttggcagccagagccaccacgaggtcaccatgtcctaccccatgcgctccgtgcagcaccggcacttccgcctcgtgcacaacctcaacttcaagatgccctttcccatcgaccaggacttctacgtctcacccaccttccaggacctcctgaaccgcaccacagctggtcagcccacgggctggtacaaggacctccgtcattactactaccgggcgcgctgggagctctacgaccggagccgggacccccacgagacccagaacctggccaccgacccgcgctttgctcagcttctggagatgcttcgggaccagctggccaagtggcagtgggagacccacgacccctgggtgtgcgcccccgacggcgtcctggaggagaagctctctccccagtgccagcccctccacaatgagctgtga

SEQ ID NO: 2 - DNA, 1509 bp, Homo sapiens
  source 1..1509: mol_type = other DNA
atgagctgccccgtgcccgcctgctgcgcgctgctgctagtcctggggctctgccgggcgcgtccccggaacgcactgctgctcctcgcggatgacggaggctttgagagtggcgcgtacaacaacagcgccatcgccaccccgcacctggacgccttggcccgccgcagcctcctctttcgcaatgccttcacctcggtcagcagctgctctcccagccgcgccagcctcctcactggcctgccccagcatcagaatgggatgtacgggctgcaccaggacgtgcaccacttcaactccttcgacaaggtgcggagcctgccgctgctgctcagccaagctggtgtgcgcacaggcatcatcgggaagaagcacgtggggccggagaccgtgtacccgtttgactttgcgtacacggaggagaatggctccgtcctccaggtggggcggaacatcactagaattaagctgctcgtccggaaattcctgcagactcaggatgaccggcctttcttcctctacgtcgccttccacgacccccaccgctgtgggcactcccagccccagtacggaaccttctgtgagaagtttggcaacggagagagcggcatgggtcgtatcccagactggaccccccaggcctacgacccactggacgtgctggtgccttacttcgtccccaacaccccggcagcccgagccgacctggccgctcagtacaccaccgtcggccgcatggaccaaggagttggactggtgctccaggagctgcgtgacgccggtgtcctgaacgacacactggtgatcttcacgtccgacaacgggatccccttccccagcggcaggaccaacctgtactggccgggcactgctgaacccttactggtgtcatccccggagcacccaaaacgctggggccaagtcagcgaggcctacgtgagcctcctagacctcacgcccaccatcttggattggttctcgatcccgtaccccagctacgccatctttggctcgaagaccatccacctcactggccggtccctcctgccggcgctggaggccgagcccctctgggccaccgtctttggcagccagagccaccacgaggtcaccatgtcctaccccatgcgctccgtgcagcaccggcacttccgcctcgtgcacaacctcaacttcaagatgccctttcccatcgaccaggacttctacgtctcacccaccttccaggacctcctgaaccgcaccacagctggtcagcccacgggctggtacaaggacctccgtcattactactaccgggcgcgctgggagctctacgaccggagccgggacccccacgagacccagaacctggccaccgacccgcgctttgctcagcttctggagatgcttcgggaccagctggccaagtggcagtgggagacccacgacccctgggtgtgcgcccccgacggcgtcctggaggagaagctctctccccagtgccagcccctccacaatgagctgtga

SEQ ID NO: 3 - DNA, 228 bp, synthetic construct
  source 1..228: mol_type = other DNA
tcgttacataacttacggtaaatggcccgcctggctgaccgcccaacgacccccggactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccg

SEQ ID NO: 4 - DNA, 199 bp, synthetic construct
  source 1..199: mol_type = other DNA
cagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaggttta

SEQ ID NO: 5 - DNA, 2210 bp, synthetic construct
  source 1..2210: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctaggaagctttcgttacataacttacggtaaatggcccgcctggctgaccgcccaacgacccccggactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgctcgaccggtcgccatgtcatgccctgtccccgcatgttgtgctttgctccttgtacttgggctttgccgagcgcgtccccggaacgcactgctgctcctcgcggatgacggaggctttgagagtggcgcgtacaacaacagcgccatcgccaccccgcacctggacgccttggcccgccgcagcctcctctttcgcaatgccttcacctcggtcagcagctgctctcccagccgcgccagcctcctcactggcctgccccagcatcagaatgggatgtacgggctgcaccaggacgtgcaccacttcaactccttcgacaaggtgcggagcctgccgctgctgctcagccaagctggtgtgcgcacaggcatcatcgggaagaagcacgtggggccggagaccgtgtacccgtttgactttgcgtacacggaggagaatggctccgtcctccaggtggggcggaacatcactagaattaagctgctcgtccggaaattcctgcagactcaggatgaccggcctttcttcctctacgtcgccttccacgacccccaccgctgtgggcactcccagccccagtacggaaccttctgtgagaagtttggcaacggagagagcggcatgggtcgtatcccagactggaccccccaggcctacgacccactggacgtgctggtgccttacttcgtccccaacaccccggcagcccgagccgacctggccgctcagtacaccaccgtcggccgcatggaccaaggagttggactggtgctccaggagctgcgtgacgccggtgtcctgaacgacacactggtgatcttcacgtccgacaacgggatccccttccccagcggcaggaccaacctgtactggccgggcactgctgaacccttactggtgtcatccccggagcacccaaaacgctggggccaagtcagcgaggcctacgtgagcctcctagacctcacgcccaccatcttggattggttctcgatcccgtaccccagctacgccatctttggctcgaagaccatccacctcactggccggtccctcctgccggcgctggaggccgagcccctctgggccaccgtctttggcagccagagccaccacgaggtcaccatgtcctaccccatgcgctccgtgcagcaccggcacttccgcctcgtgcacaacctcaacttcaagatgccctttcccatcgaccaggacttctacgtctcacccaccttccaggacctcctgaaccgcaccacagctggtcagcccacgggctggtacaaggacctccgtcattactactaccgggcgcgctgggagctctacgaccggagccgggacccccacgagacccagaacctggccaccgacccgcgctttgctcagcttctggagatgcttcgggaccagctggccaagtggcagtgggagacccacgacccctgggtgtgcgcccccgacggcgtcctggaggagaagctctctccccagtgccagcccctccacaatgagctgtgaccatcagatctcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaggtttaaaccccactccctctctgcgcgctcgctcgctcactgaggccgggcgaccaaaggtcgcccgacgcccgggctttgcccgggcggcctcagtgagcgagcgagcgcgcag

SEQ ID NO: 6 - DNA, 2210 bp, synthetic construct
  source 1..2210: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctaggaagctttcgttacataacttacggtaaatggcccgcctggctgaccgcccaacgacccccggactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgctcgaccggtcgccatgagctgccccgtgcccgcctgctgcgcgctgctgctagtcctggggctctgccgggcgcgtccccggaacgcactgctgctcctcgcggatgacggaggctttgagagtggcgcgtacaacaacagcgccatcgccaccccgcacctggacgccttggcccgccgcagcctcctctttcgcaatgccttcacctcggtcagcagctgctctcccagccgcgccagcctcctcactggcctgccccagcatcagaatgggatgtacgggctgcaccaggacgtgcaccacttcaactccttcgacaaggtgcggagcctgccgctgctgctcagccaagctggtgtgcgcacaggcatcatcgggaagaagcacgtggggccggagaccgtgtacccgtttgactttgcgtacacggaggagaatggctccgtcctccaggtggggcggaacatcactagaattaagctgctcgtccggaaattcctgcagactcaggatgaccggcctttcttcctctacgtcgccttccacgacccccaccgctgtgggcactcccagccccagtacggaaccttctgtgagaagtttggcaacggagagagcggcatgggtcgtatcccagactggaccccccaggcctacgacccactggacgtgctggtgccttacttcgtccccaacaccccggcagcccgagccgacctggccgctcagtacaccaccgtcggccgcatggaccaaggagttggactggtgctccaggagctgcgtgacgccggtgtcctgaacgacacactggtgatcttcacgtccgacaacgggatccccttccccagcggcaggaccaacctgtactggccgggcactgctgaacccttactggtgtcatccccggagcacccaaaacgctggggccaagtcagcgaggcctacgtgagcctcctagacctcacgcccaccatcttggattggttctcgatcccgtaccccagctacgccatctttggctcgaagaccatccacctcactggccggtccctcctgccggcgctggaggccgagcccctctgggccaccgtctttggcagccagagccaccacgaggtcaccatgtcctaccccatgcgctccgtgcagcaccggcacttccgcctcgtgcacaacctcaacttcaagatgccctttcccatcgaccaggacttctacgtctcacccaccttccaggacctcctgaaccgcaccacagctggtcagcccacgggctggtacaaggacctccgtcattactactaccgggcgcgctgggagctctacgaccggagccgggacccccacgagacccagaacctggccaccgacccgcgctttgctcagcttctggagatgcttcgggaccagctggccaagtggcagtgggagacccacgacccctgggtgtgcgcccccgacggcgtcctggaggagaagctctctccccagtgccagcccctccacaatgagctgtgaccatcagatctcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaggtttaaaccccactccctctctgcgcgctcgctcgctcactgaggccgggcgaccaaaggtcgcccgacgcccgggctttgcccgggcggcctcagtgagcgagcgagcgcgcag

SEQ ID NO: 7 - DNA, 5804 bp, synthetic construct
  source 1..5804: mol_type = other DNA
cagcagctgcgcgctcgctcgctcactgaggccgcccgggcaaagcccgggcgtcgggcgacctttggtcgcccggcctcagtgagcgagcgagcgcgcagagagggagtggggtttaaacctaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgagatctgatggtcacagctcattgtggaggggctggcactggggagagagcttctcctccaggacgccgtcgggggcgcacacccaggggtcgtgggtctcccactgccacttggccagctggtcccgaagcatctccagaagctgagcaaagcgcgggtcggtggccaggttctgggtctcgtgggggtcccggctccggtcgtagagctcccagcgcgcccggtagtagtaatgacggaggtccttgtaccagcccgtgggctgaccagctgtggtgcggttcaggaggtcctggaaggtgggtgagacgtagaagtcctggtcgatgggaaagggcatcttgaagttgaggttgtgcacgaggcggaagtgccggtgctgcacggagcgcatggggtaggacatggtgacctcgtggtggctctggctgccaaagacggtggcccagaggggctcggcctccagcgccggcaggagggaccggccagtgaggtggatggtcttcgagccaaagatggcgtagctggggtacgggatcgagaaccaatccaagatggtgggcgtgaggtctaggaggctcacgtaggcctcgctgacttggccccagcgttttgggtgctccggggatgacaccagtaagggttcagcagtgcccggccagtacaggttggtcctgccgctggggaaggggatcccgttgtcggacgtgaagatcaccagtgtgtcgttcaggacaccggcgtcacgcagctcctggagcaccagtccaactccttggtccatgcggccgacggtggtgtactgagcggccaggtcggctcgggctgccggggtgttggggacgaagtaaggcaccagcacgtccagtgggtcgtaggcctggggggtccagtctgggatacgacccatgccgctctctccgttgccaaacttctcacagaaggttccgtactggggctgggagtgcccacagcggtgggggtcgtggaaggcgacgtagaggaagaaaggccggtcatcctgagtctgcaggaatttccggacgagcagcttaattctagtgatgttccgccccacctggaggacggagccattctcctccgtgtacgcaaagtcaaacgggtacacggtctccggccccacgtgcttcttcccgatgatgcctgtgcgcacaccagcttggctgagcagcagcggcaggctccgcaccttgtcgaaggagttgaagtggtgcacgtcctggtgcagcccgtacatcccattctgatgctggggcaggccagtgaggaggctggcgcggctgggagagcagctgctgaccgaggtgaaggcattgcgaaagaggaggctgcggcgggccaaggcgtccaggtgcggggtggcgatggcgctgttgttgtacgcgccactctcaaagcctccgtcatccgcgaggagcagcagtgcgttccggggacgcgctcggcaaagcccaagtacaaggagcaaagcacaacatgcggggacagggcatgacatggcgaccggtcgagcggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtccgggggtcgttgggcggtcagccaggcgggccatttaccgtaagttatgtaacgaaagcttcctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 8 - DNA, 1509 bp, synthetic construct
  source 1..1509: mol_type = other DNA
atgtcttgtcccgtcccagcgtgctgtgccctccttcttgtgctcggactgtgtcgcgccagacctcgaaacgccctgcttttgttggctgatgacgggggttttgagtctggggcgtataataattctgccatcgcaacgccgcatcttgatgcgttggcaagacggtcactccttttccgaaacgcattcacaagcgtgagttcatgcagtccttcacgagcttcccttcttacagggctcccgcaacatcaaaacggtatgtatggcctccatcaagatgtgcatcacttcaatagtttcgataaggttcgatctctgccactgttgttgagtcaagccggtgtacggactggaatcattggcaaaaaacatgtagggccggaaactgtgtacccctttgacttcgcatacacggaagaaaacggttccgttctccaagttggcagaaacattacccggattaagcttttggtaagaaagttcttgcagactcaggatgaccgccctttcttcctttacgtcgccttccatgaccctcatcgatgtggtcattcacagcctcagtacgggaccttctgtgaaaagtttggaaatggagaatcaggtatgggtcggataccagactggactccccaggcttacgacccattggatgtacttgtcccctactttgtgcccaatactcccgctgccagagcggatctcgcggcccagtacacgacggtcgggcgaatggatcaaggtgttgggcttgtcttgcaagagctccgcgatgcgggagtacttaacgatacactggttatatttacatctgataatggtatacccttcccttctggacggaccaatctctactggcctggcactgcggagcccctccttgtgagttccccggagcacccgaagcgatggggccaggtatcagaagcgtatgtctccctccttgatcttactccaactatactcgactggttctccattccgtatccgagttatgcgattttcggatccaaaacaatccatttgactgggagaagtcttcttccggcactggaagccgaacctttgtgggccaccgtatttggtagtcaaagccaccacgaagtaactatgtcctaccctatgaggagtgtccaacaccggcactttagacttgtgcataacctgaatttcaagatgccattcccgattgatcaagatttctacgtatcccctacgttccaagatctgttgaataggacgacagctggtcaaccgacggggtggtataaggacctgcgccattactattatagagcacgatgggagttgtatgatagaagccgcgacccccacgaaacgcaaaacttggcgacggatccgaggttcgctcaactgctggagatgttgcgagatcagctggctaaatggcaatgggagacgcatgatccctgggtatgtgcccccgatggggtgcttgaggaaaaacttagcccacaatgtcaaccactgcacaatgaattgtga

SEQ ID NO: 9 - DNA, 1509 bp, synthetic construct
  source 1..1509: mol_type = other DNA
atgagttgcccggtcccggcctgctgcgcgctcctgcttgttttgggcttgtgtcgggctcgaccacgaaatgcgcttctcctcttggctgacgatggtgggtttgaatccggggcgtacaacaattccgccatagccaccccccatttggatgccctggccaggcgaagccttcttttccggaatgcttttacttcagtctcctcatgtagtccgagtagagcttccctgttgacgggtttgcctcaacatcaaaatgggatgtacggcttgcatcaagacgtacaccactttaacagctttgacaaggtacgatctctcccccttcttttgtctcaagccggtgtgcgaactggcataatcgggaaaaaacacgtcggcccagagacggtgtatccctttgactttgcgtacacggaggaaaacggttctgtcctgcaagttggacgcaatattactcgcatcaagctgcttgttcggaagtttctgcaaacgcaggacgaccgacccttctttttgtatgtggcgtttcacgacccgcatcggtgcggacactctcagcctcaatacggaacgttctgcgaaaaattcggtaatggagaatcaggtatgggacgcataccggactggaccccgcaagcatatgatccgttggatgtccttgtgccttattttgttcctaacacgccagctgcgcgcgcggaccttgcagcgcagtatactacagtaggaagaatggaccaaggggtgggtcttgtcctccaggaactcagagacgcaggagtacttaatgataccctggtaatttttaccagtgacaacgggattccctttccatccggacgcactaatttgtattggcctgggacagcagaaccactcctggtatccagtccagagcaccctaagcgctggggccaggtctcagaagcatatgttagtctcttggacctgacacccacaatactggattggttttctataccgtatccctcctacgcaatcttcgggtcaaaaactatccacttgacgggtagaagtttgctgccagcactcgaagctgaaccactgtgggcaacagttttcgggagccagtcccaccacgaagtcaccatgtcctatccgatgagatccgtgcagcatcgacatttcagacttgttcataatctcaatttcaagatgccgtttccaatagaccaggatttttatgtatctcccacgtttcaggacctccttaaccggactactgcaggacaacctactggatggtataaggatctgcgccattactattaccgcgcaaggtgggagctttatgatagaagccgggatccgcatgaaacgcaaaacctggccactgacccgcggtttgcccagttgctggagatgctccgggaccagttggccaaatggcaatgggagacacatgatccttgggtctgtgctccggatggtgttttggaagaaaaactgtccccacaatgtcaacctctgcacaatgaactgtga

SEQ ID NO: 10 - DNA, 1509 bp, synthetic construct
  source 1..1509: mol_type = other DNA
atgagttgtccagtgccagcctgctgtgctcttctcttggtcttgggactctgcagagcaagacctcggaacgcccttcttctcttggcagatgatggagggttcgaaagtggtgcatacaacaactccgccattgcgacgccccatctggatgcgttggccaggagatccctcctcttccgcaatgcatttactagcgtaagcagttgttctccttcacgcgcaagcctgctcactggactgccacaacaccaaaatggcatgtacggactccatcaagacgttcaccacttcaattcttttgataaggtacgctctctgccgttgctgcttagtcaagcgggcgtgagaacaggtataatagggaaaaagcatgtagggcccgagaccgtatatccattcgatttcgcctatactgaagaaaacgggagcgtcctccaagtaggacggaacataactcgcattaagctgctggtccgcaaatttcttcagacacaggacgatagaccctttttcttgtacgtggcatttcatgatccccaccgatgtggacatagtcagccccaatacggcaccttttgtgaaaaattcggaaatggcgagtccgggatggggagaatccccgattggacacctcaggcctatgatccgctcgatgttctcgtgccgtatttcgttccaaatactcctgctgcacgagcggatcttgccgcacaatatactacggtcggtagaatggaccagggagtaggtctggttctgcaagagttgcgagacgccggggtcctcaacgatacgttggtgatctttacttccgataatggtattccgtttccatctgggaggactaatctctactggccaggcacggctgagcctttgctggtctctagccctgagcacccgaagcggtgggggcaagtttctgaggcctatgtgagtttgctcgatcttacgcctacgatactggactggttcagtataccgtatccgtcatacgctatttttggcagcaaaacgattcacctcacggggcgctccttgctgccggctttggaggccgagcccctttgggcgacggtatttggtagtcagtctcaccatgaggtaaccatgtcctacccaatgcggagcgtacaacacagacatttcagactggtccacaatctgaatttcaagatgccgttccctattgaccaggatttttacgtgtcaccaacattccaggatttgcttaatcgaactactgcgggtcaacctaccgggtggtacaaggatctccggcactattactaccgcgcgaggtgggaactctacgaccgctccagagacccacatgaaacacagaacctggctacagacccgcgctttgcccaacttctcgaaatgttgcgggaccaacttgcaaaatggcagtgggagacacatgatccatgggtctgcgcccctgatggggtacttgaagaaaaacttagtccacagtgccaacctttgcataacgaattgtga

SEQ ID NO: 11 - DNA, 1509 bp, synthetic construct
  source 1..1509: mol_type = other DNA
atgtcttgtcccgttccggcgtgctgtgccttgcttctcgtactgggtttgtgtcgggcccgcccgcgcaacgctctgctccttctggccgatgatggtggatttgagagtggggcctataataattctgccatagcaacgccgcatcttgatgcactggcgcgccgatcattgttgttccggaatgcctttacaagcgtaagcagttgtagtccgtctcgggcaagcctcttgacagggctccctcagcaccagaacggaatgtatggcctccaccaagatgtccatcattttaattcttttgataaggttcgaagtctgccactgcttttgagccaggccggcgtgcgcacgggaatcataggtaaaaaacatgttggtccggagaccgtctacccctttgattttgcttatacagaggaaaatggttcagtcctccaggtagggaggaatataactaggataaaactgctggtgaggaaattcttgcagacacaggacgataggccattttttctctatgtcgccttccatgacccccacagatgcgggcactcacaaccacagtacggaaccttttgcgagaagttcggtaacggagagtctggaatggggagaatcccagattggactcctcaagcgtatgatccccttgatgttctggtcccatacttcgtccccaacacacccgcggctcgggccgatcttgcagctcaatacacaacagtaggaaggatggaccaaggagttggactcgtattgcaggaactgagagatgctggtgtactgaatgatacacttgtaatatttacctcagataatggcataccattcccctccggcaggacgaatctttattggccgggtaccgcggagccgttgctggtctccagccctgaacaccctaaacggtgggggcaggtgtctgaggcttatgtgtccctgttggacctcacccctacgatactggattggtttagtatcccatatccatcttatgccatctttggatctaagaccatccacctcacaggtcgatccttgttgccagctcttgaggcggaacccctctgggctacggttttcggatcacaaagtcatcacgaggtcaccatgagttatcccatgcgctcagtgcagcacaggcatttcaggttggtccataaccttaacttcaagatgcctttcccgattgaccaagacttctacgtgtctccgacgttccaagacttgcttaacagaactacagctggacagcctacaggctggtacaaggatctccggcactactattatagggccagatgggaattgtatgaccgctccagagatcctcacgagacccaaaacttggcaaccgaccctagattcgctcaactcttggagatgcttcgggatcagctggcgaaatggcaatgggagacgcacgatccctgggtatgtgccccagacggagtgcttgaggaaaagttgtctccacagtgtcagcctctccacaatgaactttga

SEQ ID NO: 12 - DNA, 1509 bp, synthetic construct
  source 1..1509: mol_type = other DNA
atgtcatgtccggtgcccgcttgctgcgcactgttgctggttctcgggctctgtagggcgcgacctcgaaacgcgttgctgcttcttgccgacgacgggggttttgagtcaggagcttataacaattccgctatagcgactccacatctggatgctctcgcacgacgctcattgctttttagaaatgccttcacatcagtgtccagttgtagtccctcccgcgctagccttttgaccggccttcctcagcatcagaatggcatgtatggtttgcaccaggatgttcatcattttaactcctttgacaaagttagaagtctgccactgcttttgagccaggcgggtgttcggacaggaattattggtaagaaacacgttggtcctgagactgtttatccatttgacttcgcatatactgaggaaaacggcagcgtcttgcaagtcggccgaaacatcactcggattaaacttcttgtccggaagtttttgcaaacacaagatgatcgccctttctttctttacgtagcttttcacgatccacatcggtgcggccactcccagccccagtacgggacgttctgcgagaagtttggaaatggggaaagcgggatgggtcgcattccagattggaccccacaggcgtatgatcccctcgacgtactcgttccatattttgtcccaaatacgccagcagctagggccgatttggcggcgcagtacaccacggtcggtagaatggatcagggagtcggtctcgtgcttcaggagttgcgagatgcaggcgttttgaatgatacgctcgtgatattcacttccgataacggtattccttttccaagtgggaggacaaatctttactggcctggtactgccgagccgcttctcgtctcctcccccgagcacccaaagaggtgggggcaggtgagcgaagcgtatgtcagtcttcttgacctgacccctactatactcgactggttttctatcccataccctagttatgctatttttggttccaaaaccatacacctcaccgggcggtcacttcttccagctctggaggcagaaccactctgggctaccgtgttcggatcacagagtcatcacgaggtgacgatgtcctacccaatgcgcagcgttcagcaccggcactttagactggtacacaatctgaactttaaaatgccctttccgattgatcaagatttttatgtatcacctacgttccaagaccttctgaacagaaccactgctgggcaacccaccgggtggtacaaagacctgcgccattactactacagggcgcggtgggagctctacgataggtcaagggacccccacgagactcaaaatctggctacggatccaagattcgcccagctcctcgagatgcttcgagatcaattggcaaaatggcaatgggagacgcatgacccgtgggtttgcgctcccgacggtgtgctggaagagaagctgagtcctcaatgccaacctctccataacgagttgtga

SEQ ID NO: 13 - DNA, 1509 bp, synthetic construct
  source 1..1509: mol_type = other DNA
atgagctgtcctgtgcccgcatgttgcgcattgttgctggtcctcggtctttgtcgagcccggccgcggaacgcacttctgctgctggccgacgatggtggattcgagagtggtgcctataacaacagtgcaatcgccactccgcacttggatgccttggcccgccggtcattgttgttccgaaatgcattcactagtgtttcaagctgcagtccctccagagcatcccttttgaccggactgcctcaacatcaaaacggtatgtatggtctccaccaggatgttcatcattttaacagctttgataaagtccgctcacttcctttgctcctcagccaagctggagttagaacggggattatcggcaagaagcatgttggtcctgaaacggtttacccattcgatttcgcatacacggaagagaacggcagcgtcctccaagttgggagaaatataacccggataaaattgctcgttagaaaattccttcaaacgcaggacgatcgccccttctttctgtatgtcgcatttcacgaccctcataggtgtggacatagtcagccgcagtacggaacattttgtgagaaattcggaaacggcgaaagcggcatgggtagaattcctgactggactccacaggcttacgaccctctcgatgtacttgtcccatactttgtaccgaacactcctgccgcacgagcggaccttgccgcacaatatacaacggttgggcgcatggatcagggggttgggcttgtcctccaggagttgcgcgatgccggcgtccttaatgacaccttggtcatattcacctcagataacgggatcccatttccaagtggtcggactaatttgtactggcctggtacggccgaaccgctcttggtaagcagtccagaacacccaaagaggtggggtcaggtcagtgaggcatatgtaagtctcttggatcttacaccgacgattttggactggttttcaattccttatccgagttacgccattttcggttccaagactatccatctcactggccggtcactgcttccggccctggaagcagaaccgctttgggccacggtgttcggcagccagagtcaccatgaggtgactatgtcatatccaatgcgaagcgtacaacatcggcatttccgcttggtccataaccttaactttaaaatgccttttccaatcgaccaggacttttatgtaagccctacttttcaggaccttcttaaccgcacgactgcagggcagcctacagggtggtacaaagacttgaggcactattattatcgcgcaaggtgggaactttacgacaggtctcgggacccgcacgagacacagaatctggcgacggatccgcggttcgcacaactgcttgagatgctccgcgaccagttggctaaatggcaatgggagacacacgacccttgggtttgtgctccggacggggtacttgaagaaaaattgagtccgcagtgccaaccactgcacaatgaactctga

SEQ ID NO: 14 - DNA, 1509 bp, synthetic construct
  source 1..1509: mol_type = other DNA
atgagctgcccagtgccagcttgctgtgctctccttctcgtgcttggcttgtgccgcgctaggcccaggaacgctttgctcctccttgcggatgatggcggtttcgaatctggtgcctacaacaattcagctatcgcgacgccacacctcgacgcactggctaggcgctcattgctcttcaggaatgcttttacgtctgtatcaagttgttcaccaagccgggcctccctccttacagggcttccacagcatcaaaatgggatgtatgggctgcatcaagatgtacaccattttaattcttttgacaaagtgcggtcactcccactccttctcagtcaggcgggggttcggactggaatcataggaaagaagcatgtaggccctgaaactgtttacccttttgattttgcctacaccgaagagaacggatcagtgctgcaagtaggcaggaatatcacacggataaaactgctggtgcgcaaatttcttcaaacccaagatgacagaccgttcttcctctatgttgcattccacgacccgcacaggtgcggacattcacagccgcagtacgggacgttctgcgagaagttcgggaacggcgagtccgggatgggtcggattccggattggactccacaagcttacgacccattggatgttcttgtaccctacttcgttcccaacacacctgcagctagagccgaccttgcagcccagtacaccacagtgggccggatggatcaaggtgtaggtttggtacttcaggaactgcgcgacgctggagtcctcaatgacaccttggttatcttcacgtctgacaacggaattccatttccttccgggcggaccaacctctactggcccggtactgcggaacccctgctggttagttctccagaacaccccaaacggtggggacaggtttccgaagcgtacgtctcattgcttgacctgacccctactatccttgattggttttccatcccttatccatcctacgcgatattcggttctaagaccattcatcttacggggcggtctctcctccccgcccttgaggctgaacccctctgggcgaccgtgttcggctctcagtcacatcacgaagtcacgatgagttacccgatgcggagtgtacagcatcgacatttccgcttggtacacaatttgaactttaaaatgccgttcccaatcgatcaagatttctacgtgtctcctacgttccaggacttgctcaaccgcactaccgcaggccaaccaacgggttggtacaaggatctgcgacactactattacagggcgcggtgggagttgtatgaccgctctcgggatccgcatgaaacccagaatttggcaacagatccccgatttgcccaattgctcgagatgctgagggaccaactggccaagtggcagtgggaaacgcacgacccgtgggtctgcgcgccagacggtgtccttgaggaaaaactgtccccacagtgccaacctttgcacaatgaattgtga

SEQ ID NO: 15 - DNA, 1509 bp, synthetic construct
  source 1..1509: mol_type = other DNA
atgagttgcccggtgcccgcttgttgtgcactccttcttgtgctcggcctgtgtcgagcgcgaccgaggaatgctctgctgctgctcgccgatgatgggggatttgagagtggggcgtacaacaacagtgcgattgccacccctcatcttgatgccctggctcggagatcactcttgttcagaaacgcatttacttcagtgtcttcctgctccccatcacgcgcttctcttctcacaggcctgccgcaacaccagaatggaatgtacggactccatcaggatgtgcaccatttcaactcattcgacaaagtgcggagcttgcctcttctgctctcccaagctggcgtgcgcacaggcatcatcggtaaaaaacatgttggaccggaaacagtttacccttttgactttgcgtacacggaggagaatggcagcgtgctgcaagttggtcgaaacattacacgaattaagcttctggtgcgaaaatttttgcaaacacaagatgacagaccattcttcttgtatgtagcttttcatgatccgcatcgctgtggtcactcccagcctcagtacgggacgttctgtgaaaagttcgggaatggagagtctggaatggggagaataccagattggacccctcaggcttatgatccgcttgatgtattggtcccgtactttgtccccaataccccagccgcccgagctgatctcgctgcacagtacactacagtcggccgaatggatcaaggcgtagggctcgtgttgcaagaattgcgggacgccggtgtgctcaacgataccttggtaattttcactagcgacaacggaatcccgtttccctcaggtaggactaacctgtattggcccgggacagcggaacccctgctggtcagtagccctgaacatccaaaacgctggggacaagtttccgaggcatatgtctcactgctcgatctgacccccaccattctcgactggtttagtatcccttaccctagttacgccatcttcggtagtaagactatacaccttacagggcgatctttgcttccggcacttgaagccgaaccgctctgggcgacagtctttggaagccagagtcaccacgaggtaactatgagttaccctatgcgcagtgtccaacacaggcacttccggctcgtacacaacctcaatttcaaaatgcctttccccattgaccaagacttctacgtgtccccgacgtttcaagacctgctcaataggaccaccgctggtcaaccaacgggatggtacaaggaccttagacactactactaccgcgcaagatgggagctctacgatcggtcccgggacccccatgaaactcaaaacttggccacggacccgaggttcgcccagctgctggaaatgctccgcgaccaactcgccaaatggcagtgggagacacatgatccgtgggtctgtgccccagatggggtcctggaggaaaaacttagccctcagtgccaaccgctgcacaatgaactgtga

SEQ ID NO: 16 - DNA, 60 bp, synthetic construct
  source 1..60: mol_type = other DNA
atgtcatgccctgtccccgcatgttgtgctttgctccttgtacttgggctttgccgagcg